(19) United States
(12) Patent Application Publication
(10) Pub. No.: US 2003/0105594 A1
(43) Pub. Date: Jun. 5, 2003

(54) cDNA databases for analysis of hematopoietic tissue

(76) Inventors: Carol A. Westbrook, Chicago, IL (US); Ronald Hoffman, Chicago, IL (US)

Correspondence Address: BARNES & THORNBURG, 2600 CHASE PLAZA, 10 LASALLE STREET, CHICAGO, IL, 60603

(21) Appl. No.: 10/174,513

(22) Filed: Jun. 18, 2002

Related U.S. Application Data
(63) Continuation-in-part of application No. 09/897,798, filed on Jul. 2, 2001.
(60) Provisional application No. 60/216,829, filed on Jul. 7, 2000.

(51) Int. Cl. G06F 19/00; G01N 33/48; G01N 33/50

(52) U.S. Cl. 702/19; 702/20

(57) ABSTRACT

A unique database, a “transcriptosome” of a primate CD34+ cell, was compiled which is useful for the analysis and transplantation of bone marrow. Research and clinical applications arise from analysis of bone marrow, and related hemotopoietic tissues, prior to gene therapy or transplantation. Because the database contains many unknown and uncharacterized genes, an important use is the discovery of new genes that are relevant to hematopoiesis and stem cell growth. These genes may lead to further commercial products.

### BACKGROUND OF THE INVENTION

This application is a continuation-in-part of U.S. Ser. No. 09/897,798 filed Jul. 2, 2001 which claims priority from U.S. Provisional Application Serial No. 60/216829 filed Jul. 7, 2000.

A unique database, a “transcriptosome” of a primate CD34 antigen positive cell, was compiled which is useful for the analysis of hematopoietic tissue and development of therapeutic regimes. Molecules with nucleotide sequences that are in the database may be placed in arrays on microchips for various applications or simply used as an organized group. For example, a transcription factors (TFs)/regulator proteins dataset has been extracted to explore key roles in hematopoiesis.

Although the human genome has been sequenced, meaningful groupings and uses of the sequences are just beginning. Specific purpose databases (datasets) are not available for bone marrow and related tissues.

Datasets of transcription factors that play a critical role in the process of lineage commitment and differentiation in hematopoietic tissue, would be valuable. Several such factors are known to control the basic molecular mechanisms which underlie these processes, and their expression is tightly regulated in a stage- and lineage-specific manner. For example, the level of expression of PU.1 and GATA binding proteins plays a major regulatory role in myeloid development, with PU.1 being up-regulated with myeloid differentiation, while GATA1 and GATA2 are down-regulated. Disruption in the expression, sequence or structure of critical transcription factors or their associated regulatory proteins can upset the delicate balance between proliferation and differentiation and lead to leukemogenesis. Most of the consistent translocations in myeloid leukemias that have been analyzed to date result in a fusion protein which alters the normal function of a transcription factor or a related regulatory protein. It is increasingly recognized that these genes might also contribute to leukemia by functional inactivation effected by mutation or chromosomal translocation. It has been speculated that the majority of translocations which have not yet been fully-characterized probably also involve transcriptional regulatory proteins. Thus, the identification of novel transcriptional regulators, especially those which are located near translocation breakpoints, are expected to help to specify new leukemia-related proteins, leading to better understanding and treatment of this disease.

The concept of cDNA arrays has been proposed, and various technologies are available. However, creation of databases by selecting genes according to a plan and/or specific uses or functions, for example of arrays (microarray), to put on chips, is still an active area of research. An example is the “lymphoma chip” that was recently reported, which contained arrays of genes used for diagnosis of lymphoma (Alizadeh et al., 2000).

To prepare an array of molecules so that it can be used for a specified purpose, some sort of support is generally used. For example, cDNA chips are solid supports (usually glass slides or filter membranes) containing DNA fragments from a specific plurality of cDNAs, ESTs, or control molecules organized in 2-dimensional patterned arrays, which are used for hybridization to RNA or DNA probes. The chips are used, for example, to detect the presence, as well as the relative level of expression of each DNA of the array in a target sample. The technology of cDNA arrays and of signal quantitation is developed, but specific uses of the arrays, the nature of the DNA to be placed on the chips, and medical application of chips is still under investigation. Moreover, the term “chip” is becoming broad. “Microarray” means that a plurality of very small molecules are included, regardless of the method of unified transport and use. Databases are useful to “mine” for molecules suitable for creating arrays for specific applications.

### SUMMARY OF THE INVENTION

The invention includes a database that contain UniGene and Gen Bank numbers for cDNA molecules including those for genes with known functions, in addition to genes with unknown functions, and ESTs (expressed sequence tags). The numbers refer to public databases which allow a user to find partial nucleotide sequences. The database is useful for the identification of genes relevant to hematopoiesis, and for the preparation of arrays that are capable of being organized on a microarray chip (“microchip” or “biochip”) or other physical manifestations that can be used to analyze hematopoietic tissue (bone marrow, peripheral blood, leukemia cells) for clinical applications such as bone marrow transplantation, and for research in human and other primate studies relating to hematopoiesis. Treatment regimes are a target of the invention. The unique aspects of this invention include the method in which the genes were identified as significantly expressed in bone marrow, the preliminary and expanded gene lists (the database), the concept of using the gene lists as a stem cell, transcription factors or hematopoiesis-specific database, the concept of using the gene list for a cDNA chip or other microarray, and the application of arrays for clinical and research purposes.

In an embodiment, a global approach was used to identify novel and known transcriptional regulators that might participate in hematopoiesis and leukemogenesis. A database of genes that are expressed in normal hematopoietic stem cells was surveyed. The database of 15,970 transcripts that are present in human bone marrow CD34 antigen-positive cells was searched to identify those with functional motifs consistent with transcriptional regulators. A murine stem cell database was also searched to find the human homologues of transcription factors expressed in this tissue. 330 genes were identified which are potential transcriptional regulators, including 106 transcription factors, of which 25 are novel or poorly-characterized. These transcription factors, especially those novel ones that have not been reported previously are used to discover new pathways in hematopoiesis or leukemogenesis.

Transcription factors (TFs) and the regulatory proteins that control them play key roles in hematopoiesis, controlling basic processes of cell growth and differentiation; disruption of these processes may lead to leukemogenesis. A goal of the present invention was to identify functionally novel and partially-characterized TFs/regulatory proteins that are expressed in undifferentiated hematopoietic tissue. The database of 15,970 genes/ESTs representing the normal human CD34+ cells transcriptosome (http://westsun.hema.uic.edu/cd34.html), was surveyed using the UniGene annotation text descriptor, to identify genes with motifs consistent with transcriptional regulators. 285 genes were identified. The human homologues of the transcription factors reported in the murine stem cell database (SCdb) (http://stemcell.princeton.edu/), were also identified—selecting an additional 45 genes/ESTs.

UniGene is an experimental system for automatically partitioning (categorizing) GenBank sequences into a non-redundant set of gene-oriented clusters. GenBank is an archive of published sequences. Each UniGene cluster contains sequences that represent a unique gene, as well as related information such as the tissue types in which the gene has been expressed and map location.

In addition to sequences of well-characterized genes, hundreds of thousands of novel expressed sequence tag (EST) sequences have been included. Consequently, the collection may be of use to the community as a resource for gene discovery.

An exhaustive literature search of each of these 330 unique genes was performed to determine if any had been previously reported, and to obtain additional characterizing information. Of the resulting gene list, 106 were considered to be potential transcription factors. Overall, the transcriptional regulator dataset consists of 165 novel or poorly-characterized genes, including 25 that appeared to be transcription factors. Among these novel and poorly-characterized genes are a cell growth regulatory with ring finger domain protein (CGR19, Hs.59106), an RB-associated CRAB repressor (RBAK, Hs.7222), a death associated transcription factor 1 (DATF1, Hs.155313), and a p38-interacting protein (P38IP, Hs. 171185). The identification of these novel and partially-characterized potential transcriptional regulators adds a wealth of information to understanding the molecular aspects of hematopoiesis and hematopoietic disorders.

### BRIEF DESCRIPTION OF THE DRAWINGS

FIG. 1 shows the correlation of gene expression between human and baboon CD34+ cells. The normalized intensities of all the data points (25,920) from five releases of GeneFilters (GF200-GF204) hybridized to the baboon-derived CD34+ probe were compared to those resulting from the human-derived CD34+ probe by scatter analysis, using Microsoft Excel software.

FIG. 2 lists abundance categories of the common genes in human and baboon CD34+ cells. A total of 15,407 cDNAs whose expression varies less than 3-fold between human and baboon CD34+ RNAs were arbitrarily grouped into four relative expression categories, from low to very high abundance. The categories, based on the signal intensity of the human RNA relative filter background, are as follows: no expression (<3-fold), low abundance (3-fold to <10-fold), intermediate (10-fold to <25-fold), high (25-fold to <100-fold), and very high abundance (100-fold and higher).

FIG. 3 compares the expression level between human and baboon CD34+ cells for genes selected from different abundance categories, by semi-quantitative RT-PCR. Five known genes representative of each of the abundance categories described in FIG. 2 were analyzed by RT-PCR using primers from the 3′-untranslated region of the gene. The PCR reactions were done with (+) or without (−) addition of reverse transcriptase (RT) for the indicated cycle number (Cy). The genes tested are: TM4SF4, transmembrane 4 superfamily member 4; PTK9, protein tyrosine kinase 9; CYP1B1, cytochrome P450, subfamily 1 (dioxine-inducible), polypeptide 1 (glaucoma 3, primary infantile); CSF3R, colony stimulating factor 3 receptor; β2-microglobulin. The intensity measured with GeneFilters was compared to that measured by RT-PCR.

FIG. 4 compares the expression level between human and baboon CD34+ cells for apparent species-specific genes selected from Table 3. Representative analysis by semiquantitative RT-PCR for three transcripts from Table 3 with apparent species-specific expression as measured on GeneFilters , using primers designed from the 3′-untranslated region of the gene. The PCR reactions were done with (+) or without (−) addition of reverse transcriptase (RT) for the indicated cycle number (Cy). The intensity measured with Gene Filters (GF) is compared to that measured by RT-PCR, normalized to genomic DNA. Intensity ratio measurement are shown as positive when expression in humans is higher than baboons, and negative when the reverse is true.

### DESCRIPTION OF THE INVENTION

The invention relates a database (“transcriptosome”) of a primate CD34+ cell that includes information related to nucleotide sequences that are selected by methods of the present invention as specific datasets that can be used as arrays.

Because the database contains information on many unknown and uncharacterized genes, an important use of the methods and databases of the invention is to discover new genes that are relevant to hematopoiesis and stem cell growth. The database also has value because it can be mined for specific gene discovery, for example to find new genes that are surface markers (e.g. for flow cytometry), growth factors, or receptors for growth factors that regulate stem cell growth (see Examples and Tables). The database itself may have commercial use in its entirety for the preparation of chips, which could be used to diagnose or analyze hematopoietic cancers, and to evaluate normal bone marrow or stem cells prior to transplantation.

More particularly, the invention relates to a database that is a dataset which specifies the majority of genes expressed at moderate levels or higher in human hematopoietic tissue, as represented by CD34+ cells from bone marrow, and their approximate rank order by level of expression. The genes in this database refer to partial sequences that are available in the Human Genome databases, and thus can be analyzed directly by reference to their unique ID numbers. The database has value because it can be mined to identify abundant mRNAs coding for proteins of interest in many categories with therapeutic, research, and diagnostic applications, e.g. transcription factors. The gene list, or a subset thereof, is useful to prepare a cDNA chip with applications to hematopoiesis. A transcriptional/regulatory gene dataset is in an embodiment disclosed herein

An aspect of the invention is a standard size cDNA chip (for example, having 5,000 to 10,000 elements) constructed to contain genes expressed in human bone marrow, specifically those that are expressed in the CD34+ fraction, the fraction which contains the undifferentiated cells that give rise to stem cells and which contains transplantable elements. The cDNA composition of a chip made in this fashion is representative of genes that are expressed at moderate to high levels by human bone marrow cells in their native stage (natural, in vivo), and those genes whose expression might change with physiologic or pharmacologic manipulation, as well as those genes used as internal controls. However, other compositions of cDNA molecules are within the scope of the invention.

The invention also relates the composition of a chip, that is, the selection of DNA molecules to array (position on a support in accord with a plan, or strategy) on the chip, which is based on the results of a novel experimental method (“chip” is used herein to include any kind of support for a molecular array). The invention also specifies some of the uses of the chip, which include analysis of human bone marrow, peripheral blood or cord blood prior to transplantation to determine if the transplanted tissue will engraft; analysis of human bone marrow, peripheral blood or cord blood after it has been treated with approved or experimental manipulations (e.g. growth factors, purging, gene therapy, and the like) prior to transplantation, to determine if the transplantation will engraft, or to determine the effects of treatment; research in human bone marrow transplantation and ex vivo cellular expansion; discovery of new genes related to human hematopoiesis or stem cell growth; similar research in non-human primate system, with the aim of applying the research results to human systems.

A cDNA chip called, for example, the “Stem Cell Chip” is useful as a substrate for hybridization of RNA derived from human clinical or research samples, including hematopoeitic stem cells obtained from sources such as bone marrow, peripheral blood, or cord blood; or from similar samples obtained from primate bone marrow for research purposes. The term “the chip” used hereinafter includes a plurality of chips either of similar or different compositions. Alternatively, the gene list can be mined without preparing a chip from it. The preparation of a chip is one aspect of the invention and use of the database.

For use of a chip, RNA is used to prepare a probe using standard methods (reverse-transcription, labeling by fluorescent or radioactive nucleotides), and the RNA is hybridized to the Stem Cell Chip. Hybridization occurs between homologous sequences—the degree of homology required for hybridization depends on the conditions under which the hybridization takes place, e.g., temperature, pH. Hybridization to each cDNA molecule on the array is detected and quantitated. The pattern and the relative intensity of hybridization of the probes with each cDNA on the array is expected to vary with the population tested. Individual hybridization patterns and intensity levels define “clusters” of gene expression that are used to define physiologic conditions. For example, the chip may be applied to analyze a bone marrow that was treated with gene therapy, to determine if the marrow is likely to engraft for transplantation. The expression of genes on the chip is then compared to that level of expression needed for a successful graft.

Another novel use of a chip or other form of an array is the study of experimental methods applied to non-human primates, particularly baboons. Because the chip is expected to be similarly representative of both human and baboon marrow, the use of this chip to analyze baboon marrow (stem cells or cord blood) makes it possible to directly apply the animal results to human systems. Because the chip may contain many uncharacterized gene fragments in the form of ESTs, an important use is in the discovery of new genes that are relevant to hematopoiesis and stem cell growth. Their relevancy is based on their inclusion on the gene list, and also by experimental uses of the chip such as to determine results of treatment, or comparisons of populations.

Highly-abundant genes in the transcriptosome of human and baboon CD34 included antigen-positive bone marrow cells. Non-human primates are useful large animal model systems for the in vivo study of hematopoietic stem cell biology (Andrews et al., 1992; Brandt et al., 1999; Goodell et al., 1997). To ascertain and analyze the degree of similarity of the hematopoietic systems between humans and baboons, and to explore the relevance of such studies in non-human primates to humans, the global gene expression profiles of bone marrow CD34+ cells isolated from these two species were compared. The cDNA filter arrays used (GeneFilters™) contained 25,920 cDNAs from the UniGene dataset (http://www.ncbi.nlm.nih.gov/UniGene/index.html), including both known genes and uncharacterized ESTs, permitting the survey of one-fourth to one half of the estimated 50,000-100,000 genes in the genome. The expression pattern and relative gene abundance of the two RNA sources was similar, with a correlation coefficient of 0.87. Homology was expected because they represent a marrow fraction enriched for both primitive hematopoietic stem and progenitor cells (Link et al., 1996; Pierelli et al., 2000; Ueda et al., 2000; Liao et al., 1998; Trezise et al., 1989). A total of 15,970 of these cDNAs were expressed in human CD34+ cells, of which the majority (96%) varied less than 3-fold in their relative level of expression between human and baboon. RT-PCR analysis of selected genes confirmed that expression was comparable between the two species. No species-restricted transcripts have been identified, further reinforcing the high degree of similarity between the two populations. A subset of 1554 cDNAs which are expressed at levels 100-fold and greater than background includes 959 ESTs and uncharacterized cDNAs, and 595 named genes, including many that are clearly involved in hematopoiesis. The cDNAs disclosed herein represent a selection of some of the most highly-abundant genes in hematopoietic cells, and provide a starting point to develop a profile of the transcriptosome of CD34+ cells.

The use of non-human primates permits a degree of experimental freedom to perturb hematopoiesis not possible in man, which might end in a genetic analysis of hematopoiesis, not only under steady-state conditions, but also under conditions of stress. The baboon (Papio anubis) is particularly useful in this regard because it is closely related to humans, and shows cross-reactivity with many of the reagents used to study human hematopoiesis. Recent studies have initiated a description of the overall pattern of gene expression in murine bone marrow stem cells (Nachtman et al., 2000; Phillips et al., 2000), but by contrast, relatively little is known of the expression patterns of human bone marrow hematopoietic stem cells or the baboon marrow stem and progenitor cells.

The gene lists (databases) of this invention were defined using a unique approach combining filter array methodology with cross-species hybridization to identify conserved sequences. Normal human bone marrow from an anonymous donor was fractionated into CD34+ cells by standard methods (using anti-CD34+ antibody to bind and separate out cells). RNA was prepared from the CD34+ cells so obtained, and then used to prepare a hybridization probe by radioactive labeling; the probe was hybridized to a commercially-available cDNA filter array (GeneFilters, release 200-204, purchased from Research Genetics, Huntsville, Ala.), which contained in total 25,900 cDNAs and ESTs from the UniGene set. The 25,900 genes surveyed represent ⅓ to ½ of the estimated 50,000 to 75,000 genes in the human genome. After hybridization of the arrays to the human CD34+ RNA probe, similar probes were prepared from normal baboon marrow cells that had been similarly purified for CD34+ cells. Comparison of the hybridization profiles of the human and baboon marrow made it possible to determine that both had similar expression patterns for the majority of genes. The use of a cross-species hybridization (human and baboon) ensured the selection of genes that were conserved between both species. Thus, the selected genes which are present in both RNAs are expected to be more representative of the tissue, i.e. CD34+ cells, than of the individual species. The correlation of human and baboon marrow varied from 88% to 98%, depending on the filter analyzed, with an average correlation of 94%. (To put these figures in perspective, a correlation coefficient of 0.42 was measured when comparing CDE34+ expression on GeneFilters to that obtained for the hematopoietic cell line U937 and a correlation coefficient of 0.57 when comparing human CD34+ cells to HT29 colon cancer cell line).

A set of approximately 9,500 genes was selected using two criteria: all of those expressed at similar levels in both human and baboon (which was defined as a level of expression that varied 3-fold or less between the species) and whose expression in the human was 7-fold or greater than the background level that was measured in the individual GeneFilter experiment (which was arbitrarily assigned to indicate expression at a moderate to high level). A cut-off level of intensity of 3-fold over background is generally taken to indicate expression that is greater than zero, and can be reliably detected and quantitatively measured for the human-based probes. Using this cut-off of 3-fold, the human CD34+ cells displayed approximately 15,970 or 62% of the 25,920 cDNAs present on these filters. The level of 7-fold over background was thus arbitrarily selected as a cut-off for this gene list, recognizing that all of these genes are certain to be actually expressed in the cells, and to provide a dataset that was limited in size to <10,000 genes, and contained those that are expressed at moderate to high levels; a more complete dataset would include the entire 15,970 genes; by extrapolation, this may represent half to third of all of the genes in the CD34+ cells. For some applications, different cut-off levels could be utilized—a higher cut-off would result in fewer genes but they would be a high level, and a lower cut-off would be more inclusive of the entire expression profile of the cell.

Genes from this database were then ranked from highest to lowest level of expression, as determined from their measured intensity in human CD34+ RNA. The rank order is only approximate, because the filters cannot provide the absolute level of expression, and there is experimental error in taking the measurements, but confirmatory experiments for randomly-selected genes have shown a fairly good correlation with rank order and expression measured by other methods. Additions, or corrections to the list may be made within the scope of the invention, but the underlying concept and the majority of the listed genes are as indicated herein. The complete gene list is available through a web site http://westsun.hema.uic.edu/html/expression.html. Table 2 shows selective highly-abundant EST's and partially characterized cDNAs in human an baboon CD34+ cells.

The gene filters which were used to identify the genes are commercially available from Research Genetics, but any filter array may be used. The genes themselves are selected from databases that are in the public domain (UniGene dataset, as part of the Human Genome Program. The invention is to compile a specialized database using the criteria herein for applications involving hematopoeisis (see Examples).

The genes defined in this invention are represented as UniGene cluster numbers. UniGene ( is a product of the Human Genome Program, maintained by the National Center for Biotechnology Research. UniGene contains over 40,000 entries, each of which represents a unique gene based on a composite of sequences of individual clones from cDNA libraries. The cDNA clones represented in UniGene are available for purchase from a number of repositories, including TIGR (The Institute For Genome Research, http://www.tigr.org/tdb/tdb.html). The dataset and representative clones are publicly available to any investigators, but the clones specified by this invention, and their association as a group with bone marrow and related cell types, and their expression levels, are not publicly available data.

Furthermore, there is currently no commercially available cDNA chip that has genes representative of human bone marrow stem cells and related cell types, nor is there such an extensive database which describes the constitution of genes expressed in human bone marrow. Furthermore, until the present invention, it was not possible to directly translate research results from experimental primate studies (baboon) to humans.

Characteristics and reference numbers may be arranged as:

1. Rank order (based on human expression).

2. CLUSTER ID (refers to the human Unique Gene number, or UniGene number, part of the Human Genome Program. http;//www.ncbi.nlm.nih.gov/UniGene/index.html)

3. GENBANK the GenBank number of the clone from the UniGene cluster which was placed on GeneFilters and which hybridized to the probe

4. Human expression level (measured experimentally, as normalized intensity).

5. Baboon expression level (measured experimentally, as normalized intensity).

6. Relative expression level, expressed as a ratio of human to baboon, from experimental data.

7. Title—name of gene or EST, extracted by Pathways software (Software from Research Genetics used to interpret the GeneFilters Result) from the UniGene databases.

8. Official gene name, if known.

Note that columns #2,3, 7 and 8 may be updated as the UniGene databases are updated, but they still refer to the same gene.

### EXAMPLES

### Example 1

### Use of the Hematopoetic Database of the Present Invention to Expand a Stem Cell Graft Ex Vivo

A use of the database is to determine whether a stem cell graft has the same level of gene expression as the host, or desired stem cells, in particular for genes known to be related to the success of expansion of a stem cell graft ex vivo. To do this, the pattern of gene expression in the host stem cells for genes in the database of the present invention must be analyzed. A comparison is then made of the level of expression of the same genes, in the graft. An embodiment of the invention is to compare expression levels of genes of a subset of genes either highly expressed in stem cells, or known to be predictive of stem cell graft expansion success.

### Example 2

### Use of the Hematopoetic Database of the Present Invention to Determine Whether or Not Genetic Modification Altered the Molecular Signature of Tissue

Gene therapy is used to alter or replace defective genes or to enhance the expression of specific genes.

To determine whether genetic modifications did or did not alter the molecular signature of tissue used in gene therapy, expression levels of genes in the database of the present invention are compared before and after the modifications are made.

### Example 3

### Selection of Genes From the Human CD34+ Transcriptosome Database

The 15,970-genes in the human CD34+ transcriptosome database were searched for cDNAs that encode known transcription factors, and for those containing motifs that are frequently found in transcription factors and their interacting proteins. The analysis was based on a text search of the UniGene descriptor of the clones in the CD34+ database, rather than a direct homology search of the clone sequence. UniGene is a database which automatically collects and partitions GenBank and EST sequences into a non-redundant set of gene-oriented clusters by establishing sequence overlaps; each cluster represents a single potential transcript. Each cluster is annotated with a descriptor of the transcript that is the result of automated searches for sequence homologies to proteins from 8 organisms, using both nucleotide and protein sequence alignment; thus, a fair amount of functional prediction is available for each gene cluster even if it represents EST sequence that has not been further studied. Each cluster is assigned a chromosomal location, based on sequence alignment. (Details of the construction and updating of the UniGene database are available at http://www.ncbi.nlm.nih.gov/UniGene/).

The UniGene cluster descriptors contained in the CD34+ transcriptosome database were searched for terms that are thought likely to annotate transcription factors, co-repressors or co-activators, nuclear factors, and other DNA-interacting proteins. The resulting genes were updated, corrected for redundancy, and verified through homology screens. The database was visually inspected, and an additional 6 genes of known function which clearly did not contain transcriptional regulatory activity were removed from the database. A total of 285 genes resulted. Table 4A presents these genes, categorized according to their function or functional motifs, with their UniGene number, chromosomal location, and UniGene descriptor. The cDNAs in each category are presented in the order from highest abundance to lowest, based on the measured level of expression in CD34+cells.

### Example 4

### Selection of Genes from the Murine Stem Cell Database

The transcription factor category of the murine hematopoietic stem cell database was analyzed to identify the human homologues of known and novel transcription factors expressed in human bone marrow CD34+ cells, by cross-referencing the murine and human UniGene databases. The murine UniGene clusters corresponding to each of the 161 transcription factors listed in the murine database were matched with the human clusters in the UniGene database version 129 resulting in 155 homologous human clusters. A total of 145 human genes remained after updating to UniGene version 135 and removing redundant entries. Of these 145 clusters, 87 were represented in the human CD34+ transcriptosome database, including 30 which had already been identified by a search using text descriptors. These 30 clusters are indicated with an asterisk (*) in Table 4A. Analysis of the remaining 57 human genes for homology to their assigned UniGene cluster or to a corresponding TIGR entry, and excluding those whose known function was obviously not in the category of a transcriptional regulator, resulted in 45 additional genes/ESTs. These additional 45 genes are listed in Table 4B, and each entry includes the murine gene and its presumed human counterpart, its human UniGene cluster ID and descriptors, its chromosomal location, and the level of expression in human CD34+ cells. Of the 58 clusters which are not present in the CD34+ transcriptosome database, 38 were thought to be unexpressed in human CD34+ cells, based on an expression level less than 3-fold over background in the CD34+ transcriptosome database, and the remaining 20 could not be evaluated since they had not been included in the original expression studies which resulted in the CD34+ transcriptosome database.

### Example 5

### Literature Analysis of the Transcription Factor Database

After combining the datasets mined from the human and murine databases, the total number of potential transcription factors or regulatory proteins was determined to be 330. This includes 106 genes that are recognized as transcription factors, and 224 genes in other categories which include zinc fingers (90 genes); enhancers (14 genes); activators (8 genes); forkhead (11 genes); oncogenes (20); ring finger (16 genes); the combination of helix-loop-helix, homeobox, leucine zipper, nuclear, PHD, POU, and repressor categories (21 genes). The remaining 44 cDNAs represent genes which are functionally characterized as transcriptional regulators but lacked any search terms used in the mining protocol. A literature search of each of these 330 genes was performed to determine what was known about each one, emphasizing the discovery of novel genes. The following convention was used to summarize the search results: K=known gene, well-characterized; PC=partially-characterized, the gene was reported and some preliminary studies have been performed to indicate its function; N=novel gene, no functional information other than its chromosomal location and sequence homology to a known gene or gene family has been reported. These summaries are given in Tables 4A and 4B. As a result of the literature search, 165 (50%) of the 330 transcriptional regulators identified were found to be known genes, 86 (26%) have been partially-characterized, and 79 (24%) are novel. The partially-characterized and novel transcriptional regulators have been further categorized by their relative level of abundance in CD34+ cells, with 92 expressing at low level (>3-fold to <10-fold over background), 27 expressing at intermediate level (≧10-fold to <25-fold), 28 at high level (≧25-fold to <100-fold), and 18 expressing at very high levels (≧100-fold), using the conventions reported in the CD34+ transcriptosome database.

Novel transcription factors were studied. Based on a literature search of the 106 identified transcription factors, 78 appear to be well-characterized, known genes, while 18 have been partially-characterized and 7 represent truly novel genes. These 25 partially-characterized and novel genes are listed in Table 5 along with details of their presumed function and the related literature citations.

The human CD34 + transcriptosome database was prepared by hybridization of filter arrays, selecting transcripts that are common to both human and baboon bone marrow CD34+ antigen positive cells. This database is felt to be an accurate portrayal of the transcriptosome of the CD34+ cell, and was estimated to contain 50-75% of the transcripts expressed in this tissue. This database contains 15,970 genes/ESTs expressed in CD34+ cells, and lists their relative level of expression; random sampling of selected transcripts verified (by semi-quantitative reverse transcriptase PCR) that most were expressed at the predicted level.

The murine database (http://stemcell.princeton.edu/) was the result of a cDNA library study, subtracting a stem cell depleted (AA4.1^{neg}) cDNA library from a mouse fetal liver hematopoietic stem cell (Sca^{Pos}AA4.1^{Pos}Kit^{Pos}Lin^{neg/lo}) cDNA library. The subtracted library represents genome-wide gene expression in mouse hematopoietic stem cells devoid of housekeeping genes. Sequence information on each of these clones was compared by BLAST against GenBank non-redundant protein and nucleotide databases, the EST database, Swissprot, and mouse and human DOTS contigs. Each clone was categorized according to its sequence homology to genes of known functions, resulting in a “transcription factor” category containing 161 entries.

This gene list is useful for further studies of normal and malignant hematopoiesis. One of the most striking features of this list is that many of the genes have been assigned functional roles in numerous other tissues besides bone marrow. Also of note is the identification of 165 partially-characterized and novel genes, 11 of which are expressed at a very high level in CD34+ cells, suggesting that they have an important role in this tissue but have not been previously recognized as such. Some of the interesting novel or partially-characterized genes include zinc finger protein 161 (ZFP161, Hs.156000), a cell growth regulator protein with a ring finger domain (CGRl9, Hs.59106), zinc finger protein 198 (ZNF 198, Hs.109526), RB-associated CRAB repressor (RBAK, Hs.7222), death associated transcription factor 1 (DATF1, Hs.155313), and a p38-interacting protein (P38IP, Hs. 171185). The human ZFP161 protein is highly homologous (98%) to ZF5, a putative murine repressor for MYC, with a growth-inhibitory function. Both ZFP161 and RBAK may be associated factors for two very functionally important proteins, MYC and RB respectively, and may play important regulatory roles in cellular functions such as proliferation, differentiation, and apoptosis; to our knowledge, these genes have not been previously evaluated in hematopoiesis or leukemia. Another interesting protein is zinc finger protein 198 (ZNF 198). This gene has not been functionally characterized, but it is reported to be involved in the t(8; 13) translocation, resulting in a fusion protein with fibroblast growth factor receptor 1 (FGFR1).

### MATERIALS AND METHODS

I. Collection and Selection of CD34+ Marrrow Cells

Healthy adult baboons (Papio anubis) weighing 9-10 kg were used. The animals were housed under conditions approved by the Association for the Assessment and Accreditation of Laboratory Animal Care. Bone marrow aspirates were obtained from the humeri and iliac crest of adult baboons under ketamine and xylazine (1 mg/kg) anesthesia under guidlines established by the Animal Care Committee of the University of Illinois at Chicago. Human bone marrow aspirates from the iliac crest were obtained from normal human adult donors after informed consent was obtained, as approved by the Institutional Review Board of the University of Illinois at Chicago. Marrow mononuclear cells were isolated from the marrow as previously described (Brandt et al, 1999). Briefly, the marrow was heparinized; diluted 1:15 in phosphate-buffered saline (PBS); and fractionated over 60% Percoll (Pharmacia LKB, Uppsala, Sweden) by centrifugation at 500 g for 30 minutes at 200° C. The interphase mononuclear cells were resuspended in PBS containing 0.2% bovine serum albumin and human immune globulin (Sigma Chemical Co, St. Louis, Mo.) and labeled with the biotin conjugated mouse anti-human CD34+ antibodies MoAb 12-8 (Andrews et al., 1986) for baboon, and QBAND/10 (Brandt et al., 1998) for human cells, washed, and relabeled with streptavidin conjugated rat anti-mouse antibody-containing iron microbeads (Miltenyi Biotech, Auburn, Calif.). The CD34+ cells were then selected by passing the CD34+ cell-antibody-iron bead complex through a magnetic column. The purity of the CD34+ fraction was estimated by flow cytometry using a fluorescein isothiocyanite (FITC)-conjugated anti-human CD34+ antibody K6.1 (Brandt et al, 1999) for baboon cells and MoAb HPCA-2 for human cells.

II. RNA and DNA Preparation

Total RNA was extracted from 1-5×106 human and baboon CD34+ cells using an Ultraspec RNA Isolation kit (Biotecx Laboratories, Inc, Houston, Tex.) according to the manufacturer's protocol. The quantity of total RNA was determined by A260 absorbance, and quality was verified by analysis on 1% agarose gels using standard techniques. Genomic DNA was prepared from the HL60 human cell line (American Type Culture Collection) and baboon peripheral blood cells using Trizol reagent (Life Technologies) according to the manufacturer's specification.

Uniformly-labeled cDNA probes were prepared from 3 mg of total RNA by priming with 2 mg of oligo-dT, followed by elongation with 1.5 units of Superscript II reverse transcriptase (Life Technologies, Grand Island, N.Y.) in presence of 100 mCi of 33P dCTP (Amersham Pharmacia Biotech, Piscataway, N.J.). The labeled probe was purified from unincorporated nucleotides and other small molecules with ProbeQuant G-50 (Amersham Pharmacia Biotech).

III. Hybridization of cDNA Probes to GeneFilters

Five releases (GF200-204) of human GeneFilters (Research Genetics, Huntsville, Ala.) were pre-hybridized for 2 hours at 420 C. in MicroHyb solution (Research Genetics), with the addition of 1 μg/ml each of polyA (Research Genetics) and human Cotl DNA (Life Technologies, Grand Island, N.Y.). The blots were then hybridized overnight in the same MicroHyb solution with the addition of 2×106 cpm/ml of heat denatured probe. The blots were washed twice at 500 C. with 2× SSC, 1% SDS for 20 minutes and once at room temperature in 0.5× SSC, 1% SDS with gentle agitation for 15 minutes, prior to imaging. For re-use of membranes, the filters were stripped in 0.5% SDS for 1 hour at room temperature with gentle agitation as recommended by the manufacturer, and was re-exposed to confirm complete stripping.

IV. Exposure, Imaging, and Analysis of Filter Membranes

The hybridized filters were imaged using a phosphor imaging screen (Molecular Dynamics, Sunnyvale, Calif.), exposed for three to four days, imaged using a Storm phosphor imaging system (Molecular Dynamics) at 50-micron resolution, and analyzed using PathwaysII from Research Genetics following the manufacturer's guidelines. Using this program, individual cDNA spots were identified and fit to a grid, and their intensity measurements were recorded as raw intensities. The background for a particular experiment, provided as a reference, was calculated by averaging the measured intensities between the two grids of the filter. This background information was used to assign levels of expression of the genes. Data from poor hybridizations, such as those which had unacceptably high background or non-uniform control spots intensities across the membrane, was not considered for further analysis and discarded. To compare expression of a cDNA spot between two probes that were sequentially hybridized to the same filter, the intensities were normalized using the algorithm provided by the PathwaysII software, using either control spots or all data points as reference. The data were exported as Excel files for further analysis. Since PathwaysII utilizes an older, somewhat outdated version of UniGene (build versions 18, 19 ,39, and 42) and substantial changes have been made in the UniGene database since then, the cDNAs list was updated using UniGene build version 118 as reference (current as of April, 2000). To accomplish this, both the UniGene and GeneFilter dataset were reformatted to Microsoft Access database. The GenBank accession numbers of the GeneFilter dataset were then matched against the UniGene database to update the cluster ID, gene name, and gene description.

V. PCR Analysis

For reverse-transcriptase PCR (RT-PCR), first strand cDNA was generated from approximately 1 mg of RNA that had been DNase-treated with RNase free DNase I (Life Technologies, Grand Island, N.Y.). The RNA was then used to make first strand cDNA in a 20 ml reaction volume with (+RT) or without (−RT) reverse transcriptase using Superscript II Reverse Transcriptase kit from Life Technologies according to the manufacturer's recommended protocol followed by RNase H treatment. If not stated otherwise, 1/20th volume of the +/−RT reaction mix was used for the PCR reaction in presence of IX PCR buffer (Perkin Elmer Cetus (PE)), 1.5 mM MgCl2, 200 mM dNTPs, 1 mM each of forward and reverse primers, and 1U of Amplitaq polymerase (PE ) in a 20 ml reaction volume using the following cycles; initial denaturation at 950 C. for 5 min. followed by each cycle at 950 C. for 30 sec., annealing at 580 C./650 C. depending on the primer pair for 30 sec., amplification at 720 C. for 30 sec., the final amplification was for 5 min at 720 C. PCR analysis of genomic DNA was similarly performed, using 200 ng of genomic DNA instead of first strand cDNA.

VI. Comparison of Expression Levels by Semi-quantitative RT-PCR

To compare the expression of individual genes, RT-PCR was performed using primer pairs designed based on the sequence of the cDNA clones that was included on the GeneFilter. The PCR was done from 25 to 40 cycles with increments of 5-cycles, except for β2-microglobulin, which was done at 18, 22, 25, and 30 cycles. The PCR reaction products were analyzed on a 3% agarose gel stained with ethidium bromide, and the amount of DNA was quantitated as band intensities using GelDoc software from BioRAD (Hercules, Calif.). The level of expression of each gene was normalized against the level of β2-microglobulin expression between these two species. The relative expression between human and baboon cDNA was estimated by measuring the ratio of intensity of DNA product, comparing only those measurements which fell within the linear range of PCR amplification cycles; multiple determinations, when performed, were averaged. The sequences of Forward (F) and Reverse (R) primers are:

Transmembrane 4 superfamily member 4 (TM4SF4),
F-AAGCGATTTGCGATGTTCACCTC,
R-GAGGCTCTCGGCACTTGTTCC;
Protein tyrosine kinase 9 (PTK9),
F-GATTCCTTTGTTTTACCCCTGTTGGAG,
R-TTGCTGC ATACAACATTTTTTGAC;
Cytochrome P450, subfamily I (dioxin-inducible),
polypeptide 1 (glaucoma 3, primary infantile)
(CYP1B1),
F-GTAATGGTGTCCCAGTATAA GTAATGAG-3′,
R-TCATGAATGCTTTTAGTGTGTGC-3′;
Colony stimulating factor 3 receptor (granulocyte)
(CSF3R),
F-CTGAAGTTATAGGAAACAAGC ACAAAAGGC,
R-GCCC ATGACTAAAAACTACCCCAGC;
Beta-2-microglobulin (B2M),
F-CCTGAATTGCTA TGTGTCTGGG,
R-TGATGCTGCTTACATGTCTCGA.
R82595,
F:GCTCGTAGCAACATTTTCGTAATAGCC,
R:GGACCCATCGTGGTT ACCGTG;
AA676327,
F-ATATTTCGGTAACTTTTGACCCTAAG,
R:CAGGGGCAA TTTTGAGGTATG;
R85439,
F:GGCAGGGCTCTAAATGGAAGTAGTTG,
R:CTCAGAAGTGTTTTGTAGCAAGGCTGC,
AA487912,
F:AAACAGTGACTTATCCCGCTAC CC,
R:GGGTGGGTTTACTCTTAGAATCGC;
N25920,
F:CAGATGGAGGGTTTATGAGTGAGGCTGG,
R:GCTTGTTCTTTGGGGATTGTGGTGC;
R05886,
F:taggcgtgagaagcatatagaggc,
R:agtgaataagcaagaaatcagggtg;
N74363,
F:ACAAAGGGCTGTTTACTGAGAGACCTGAGC,
R:GGCATAACTCACACCCATT TGTTTACCTGC;
N55359,
F:GGCAGAATCTACTGGGCATCTTGTAATC,
R:AGTTTTGGTGGTCCAGGGAAGGTAC.

VII. Correlation of Gene Expression Between Human and Baboon CD34+ Cells

CD34+ cell populations were isolated from bone marrow aspirates by immunomagnetic cell sorting using antibodies that represent the best selection of undifferentiated and multi-potent marrow cells in human and baboon marrow. The human marrow cell population was 90% pure, as determined by FACS analysis with anti-human CD34+ antibody. Using the same method, the baboon CD34+ cells measured 77% purity. This measurement in baboon cells is an underestimate of the true degree of purity due to the relative non-specificity of the anti-human CD34+ antibody K6.1 (used for quantitation by flow cytocytometry) with baboon cells, resulting in a weaker fluorescence signal and lower estimates of purity than can be measured in comparable human cells, but it is within the range that we normally observe with this method.

Radioactively-labeled RNA-based probes prepared from each cellular population were hybridized to five nylon filter membrane arrays (GeneFilters releases 200-204, containing a total of 25,920 cDNAs) and phosphoimaged, and the resultant image was analyzed to determine the relative hybridization signal intensity for each cDNA with each probe. Each cDNA on the array is derived from a single clone from the IMAGE consortium (http://image.1lnl.gov) representing the 3′-end of a unique UniGene cluster. All data were obtained by sequential hybridization to a single filter set, in order to provide the most accurate comparisons between probes and avoid variability in cDNA spotting. Duplicate experiments were performed when possible, but were limited by the lifetime of the filters, which in general could be successfully re-hybridized no more than 3 to 4 times. It was not possible to use pooled baboon marrow donors because of the limited availability of animals, and thus pooled human donors were not used either, recognizing that the methods of the present invention are not sensitive enough to detect small differences between individual donors.

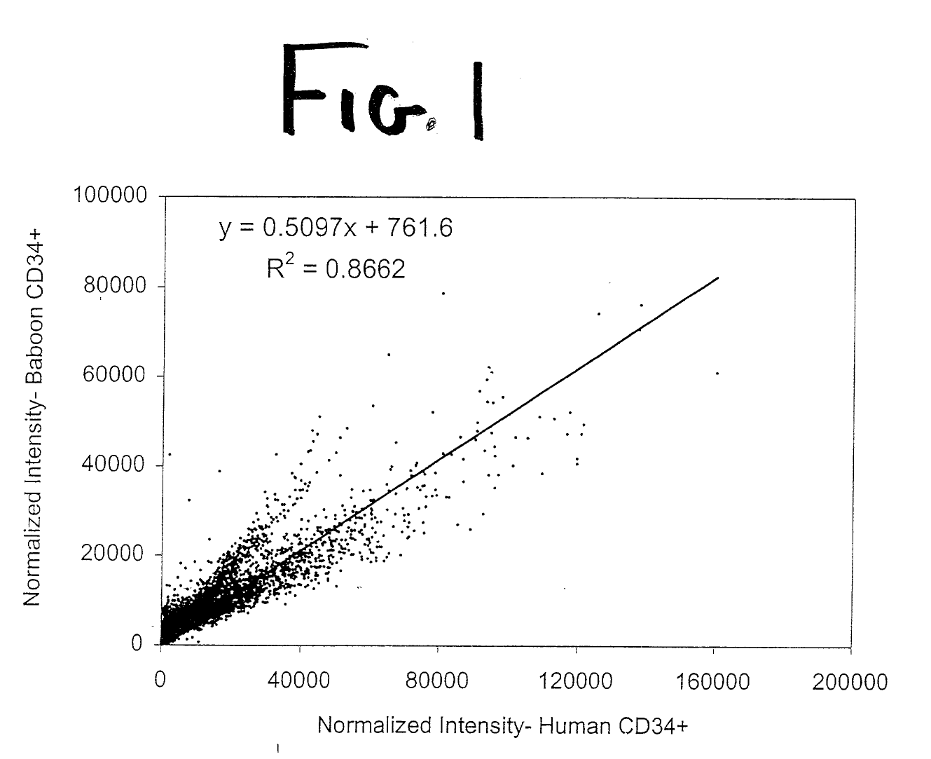

Normalized signal intensities for individual cDNA spots from these hybridizations were compared by scatter analysis, and revealed that the gene expression patterns in human and baboon cells were very similar, with an overall correlation of 0.87. The composite data for all hybridizations is summarized on a scatter plot (FIG. 1). The measured raw intensity of the hybridization signal relative to the filter background is used as an indicator of the relative abundance of the cDNA. For these experiments, a cut-off level of raw intensity (non-normalized) of 3-fold over background was used to indicate that a gene is definitively expressed in human cells. By this criteria, human CD34+ cells displayed positive expression for approximately 15,970 (62%) of the 25,920 cDNAs present on these filters. This gene list excludes many housekeeping genes, which are measured on the GeneFilters as hybridization controls but are not included for normalization by Pathways II software. (For information on all the spotted cDNA for each filter including the housekeeping genes, refer to the Research Genetics's ftp website,

The baboon-derived probes showed a consistently higher hybridization background, approximately three-fold higher, than the human-derived probes, so it was not possible to apply the same cut-off level for this species (baboon). However, 13,447 cDNAs (84%) gave a signal with the baboon probe that varied less than 2-fold from the human level of expression, while almost all of the genes (15,407 or 96.5%) were expressed within 3-fold of each other. Much of the measured differences in expression level is likely to be due to experimental variation; about 3% of cDNAs will vary more than 3-fold upon repeat hybridization with these probes. Other measured differences between the human and baboon RNAs probably reflect true differences in expression, but in either case, the variation is not great. Thus human and baboon CD34+ cells express virtually the same spectrum of genes, with similar though not identical levels of expression.

VIII. cDNAs Highly Expressed in Both Human and Baboon

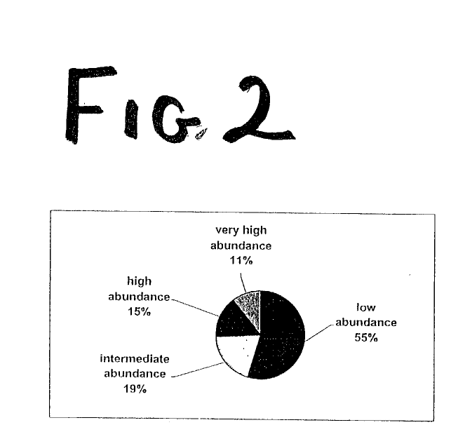

The 15,407 cDNAs that are commonly expressed in human and baboon CD34+ cells were arbitrarily placed into several groups (FIG. 2) based on their spot intensities relative to background in the human data set: very high abundance (100-fold and over), 1,619 cDNAs; high abundance (25-fold to <100-fold), 2,376 cDNAs; intermediate abundance (10-fold to <25-fold), 2,976 cDNAs; low abundance (3-fold to <10-fold), 8,436 cDNAs.

The very highly-abundant genes identified by Pathways II analysis were then updated to the most current UniGene release (version 118, April 2000), and examined in detail. A total of 1,554 UniGene clusters remained after updating. This list included 595 named genes, and 959 ESTs and uncharacterized cDNAs. This list of highly-abundant genes and ESTs is available as an appendix to the online version of this article, and is also available on our hematopoietic stem cell website (http://westsun.hema.uic.edu/html/expression.html). The named genes represent a wide variety of functional categories such as growth factors and cytokines, receptors and cell surface molecules, intracellular signalling molecules, cell cycle proteins etc. A sample of these genes, sorted by functional category, are given in Table 1. Note that this list includes many of the genes (typed in bold) that would be expected to be present in CD34+ cells, such as receptors for IL3 and colony stimulating factor 3. Interestingly, many expected hematopoietic genes are not in this category, as their level of expression is relatively low; for example, the CD34 antigen is expressed at a relatively low level, only 6-fold above background (for human).

A large fraction, over 61% of these highly-expressed cDNAs, are ESTs and uncharacterized cDNAs. Although many of these genes are uncharacterized, the UniGene database provides some information about their similarity to known proteins. Furthermore, many of the named genes represent full length cDNAs that have not been fully studied or are only partially characterized, though some function is suggested by homology to known proteins. A partial list of some of these interesting ESTs and partially characterized named genes are given in Table 2. Further characterization of the ESTs in this database represents a potential wealth of new information about the CD34+ transcriptosome.

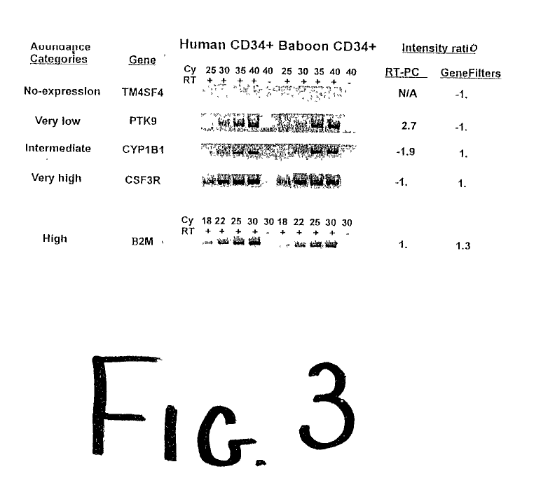

Several known genes from each abundance category were selected to verify their relative level of expression in both species by semi-quantitative RT-PCR. Representative examples are shown in FIG. 3. Each gene tested was found to be expressed at comparable levels in both species, although the abundance category was not always accurate, especially in the lower abundance genes. For example, PTK9 is expressed at a level 5-fold above background in human cells, but its signal appears stronger than CYPB1, measured at 20-fold above background. The measurement of the absolute level of expression of a cDNA using filter hybridization is related to many factors, including the amount of DNA placed on the filter (which cannot be accurately controled), and the efficiency of hybridization. Thus, the assignment of a gene to a relative abundance category can only be regarded as approximate, and may require additional confirmation.

IX. Species-specific Transcripts

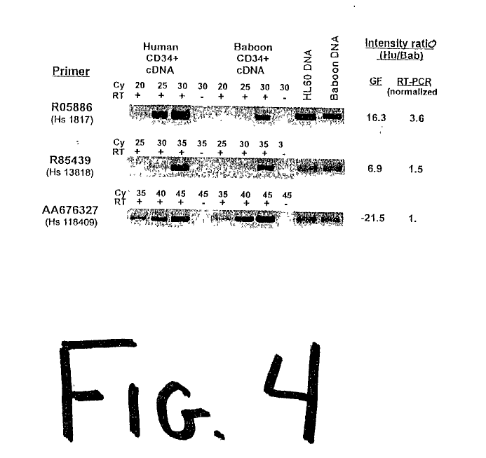

Although there were a number of cDNAs which did not appear to be highly-correlated (that is, their expression varied more than 3-fold between species), there were a few genes whose measured intensity suggested that they were preferentially expressed in only one species. To identify these genes, the GeneFilters dataset was searched for cDNAs which were unexpressed in one species (defined as a raw intensity of less than 3-fold background), and were clearly expressed in the other species (>3-fold background) with a normalized intensity ratio of >3 fold between species. There were only 14 cDNAs which fit this criteria, 6 baboon and 8 human, which includes 6 known genes and 8 ESTs. PCR primer pairs for all 14 cDNAs were designed to match the sequence of the human clones which were present on the filter membrane; the pairs were tested for their ability to amplify both genomic DNA and reverse-transcribed RNA from both species. Six primer pairs (4 human and 2 baboon) were successfully validated on both species in this manner, and these were further analyzed by semi-quatitative RT-PCR, using an additional normalization factor for PCR efficiency on genomic DNA from both species. The ratio of expression for each gene, as measured by semi-quantitative RT-PCR, is compared to that measured on GeneFilters, is summarized in Table 3, and representative examples are shown in FIG. 4. The use of normalization factors, one as a control for PCR efficiency of human-specific primers against baboon, and another for RT-reaction, adds complexity and probably some inaccuracy in quantitative comparison of gene expression between the two species, so the measured levels can only be regarded as estimates. Nonethless, most of the genes, except for two designated by Unigene Cluster ID Hs.1817 and Hs.215595, showed little if any differential between the two species and fall within 3-fold of each other, well within the arbitrary cut-off that was set for Table 1. Only Hs.1817 and Hs.215595 were confirmed to be expressed at somewhat higher levels in human than baboon (3.6-fold and 5.4-fold, respectively), although the differences were small and not as great as was measured on the filters. The results showing differential expression of Hs.1817 are included in FIG. 4. Thus, none of the 6 genes tested showed expression restricted to one species, though some appear to be differentially expressed. This result suggests that the experimental variation in the GeneFilter hybridization system is greater than the actual variation between the two species. Additional work will be required to determine if there are any bonafide species-specific genes within either species.

By its ability to simultaneously detect and quantitate the expression level of thousands of genes at one time, cDNA array technology is greatly improving our understanding of the complex patterns of gene expression in eukaryotic cells. In the present invention this technology is used to profile the gene expression patterns of CD34+ marrow cells in human and baboon cell populations. Baboon-derived probes are suitable for use on human cDNA arrays with some limitations.

Expression studies on cDNA arrays require a fairly large number of cells to isolate an appropriate amount of RNA for probe preparation. Because of this constraint, it was necessary to purify the CD34+ cells by immunomagnetic columns rather than FACS, which would require prolonged sorting. The stress imposed by the prolonged sorting time required to prepare this number of cells can dramatically reduce cell viability and yield of CD34+ cells, and may alter their gene expression profile. Because of the weak cross-reactivity of anti-human CD34+ antibody against baboon CD34+ antigen, it is difficult to accurately determine the level of purity of baboon CD34+ cell population. Thus, the purity of baboon CD34+ may be an under-representation. At any rate, in spite of the heterogeneity of the cell populations examined and the limited number of subjects studied, we determined that bone marrow cells derived from the two closely-related species have similar patterns of gene expression. Although many molecular similarities were expected between human and baboon CD34+ cells, the results suggest that the transcriptosomes are nearly identical, supporting experimental studies over the years which have demonstrated similar biologic activity. Inability to identify any species-specific transcripts further supports the similarity of the two populations.

The probe derived from the 3′ end of baboon RNA recognized human cDNAs fairly well under appropriate hybridization conditions. The concentration of Cot1 and oligo-dT which are used for blocking non-specific hybridization were found to be very crucial for this purpose. This is not unexpected, because the genomes of the two species are highly conserved, and both have Alu sequences (Hamdi et al., 2000; Hamdi et al., 1999). In general, higher background resulting from the baboon probe may be a reflection that the Alu content is not identical, and might benefit from a readjustment of the hybridization conditions, especially Cot1 and oligo-dT concentration. Nonetheless, the hybridization signal obtained with the baboon probe was strong and resulted in a very similar pattern to the one obtained with human probe. This suggests that human cDNA arrays are accurate substrates for baboon experiments, thereby facilitating translation of experimental results with this animal model to human relevance.

The studies were performed using a cDNA filter array system and radioactive probes. Although there may be limitations to the use of filters rather than solid cDNA supports, GeneFilters were especially attractive for these studies because they contain over 25,000 different cDNA clones, which covers an estimated 50% of the human genome, including a large proportion of uncharacterized cDNAs (ESTs).

The use of GeneFilters dictated an experimental design that differs from those using cDNA arrays on solid supports. Because two probes cannot be simultaneously hybridized and compared in a single experiment, reproducibility is maximized when the same membrane is re-used for sequential hybridization to compare probes from different RNA sources. Due to limited membrane lifetime, it is not possible to repeat multiple experiments, or compare expression patterns among different subjects, so the sampling error may be greater than for other methods for cDNA analysis. Thus, the results presented here should be regarded as a starting point for further confirmation and analysis.

The most reliable data obtained on these filters is the comparison of relative signal strength for a single gene between two probes. An absolute determination of the relative expression between different genes on one filter is less reliable, because the signal strength is dependent on many factors, such as the length of the clone and the hybridization efficiency of the probe, and the relative inaccuracies of spotting small amounts of DNA. Cross-comparisons of cDNA on different filters is less reliable. Here, the intensity of the hybridization signal relative to background was used as a means of comparison between filters, in order to estimate the relative level of expression of all of the genes on this dataset, recognizing that this is only an approximate-though generally reliable-measurement.

The gene list resulting from this study represents a selection of some of the most highly-abundant genes in hematopoietic cells, and provides a starting point to develop a profile of the predominant cDNAs that define CD34+ cells. Interestingly, a significant fraction of the genes identified on these filters are not unique to hematopoietic cells, but are present in other tissues. This reinforces the concept that a tissue is defined not only by the expression of tissue-specific genes, but also by the overall pattern and relative abundance of the sequences which are more widely expressed. Perhaps the most interesting result is the fact that many of the cDNAs expressed at high level in these cells have not yet been identified or characterized. The gene and EST list presented here, and their relative expression levels, represent a potential wealth of new information about bone marrow stem cells and hematopoietic progenitor cells.

A comprehensive description of the CD34+ transcriptosome with reference to the UniGenes represented in GeneFilters will be useful. Although by no means complete, the list of over 15,000 cDNAs disclosed comprises an estimated 25-50% of the genes expressed in CD34+ cells, and also provides an approximation of their relative abundance. This gene set will be useful for the production of customized cDNA arrays for bone marrow studies.

X. The Human CD34+ Transcriptosome Database

The database, which is available online at http://westsun.hema.uic.edu/cd34.html, contains 15,970 cDNAs expressed in CD34+ cells, and includes the GenBank accession number, the UniGene cluster identification number (http://www.ncbi.nlm.nih.gov/UniGene/) to which the GenBank clone belongs, its relative expression in CD34+ cells, the gene name, a functional description of the gene (from the UniGene text descriptor, build version 129), and its chromosomal location. The UniGene text descriptors of this database were searched for the following terms: transcription factor, leucine zipper, zinc finger, ring finger, helix-loop-helix, PHD, POU, forkhead, bromodomain, homeobox, oncogene, nuclear, activator, and repressor. The dataset was then updated to the most recent UniGene Build version (version 135, June 2001). Redundant cDNAs contained within the same UniGene cluster were removed, saving only the clone having the highest expression level in the CD34+ database. Each cDNA sequence was then used to search the GenBank NR database (http://www.ncbi.nlm.nih.gov/entrez/query.fcgi?db=Nucleotide) using BlastN alignment software, to verify homology to the predicted gene, using an arbitrary cut-off of E <−40 to indicate sufficient sequence identity. If the E value was greater than e−40, then the cDNA sequence was also used to search the TIGR database (http://www.tigr.org/) to verify that it is represented by a TIGR contig containing the transcript of the predicted functions. cDNAs, which did not pass these GenBank or TIGR screens, were removed from the database, as were genes of known function that did not appear to represent the categories which were being sought.

XI. The Murine Stem Cell Database

A murine stem cell database (http://stemcell.princeton.edu) consisting of expressed genes (devoid of housekeeping genes) in mouse fetal liver stem cells has been reported by Phillp et al. (2000). The GenBank accession number of all 161 entries in the “transcription factor” category of this database were selected, and used to identify the corresponding murine UniGene cluster for each entry (build version 129); the UniGene annotation was used to identify the human homologue, if available. All human genes which were also present in the CD34+ transcriptosome database were then selected for inclusion in the present study, and updated to their corresponding entry in build version 135 of UniGene. GenBank and TIGR homology screens were performed as described above.

### DOCUMENTS CITED

Ahuja H, Hong J, Aplan P, et al. t(9;11)(p22;p15) in acute myeloid leukemia results in a fusion between NUP98 and the gene encoding transcriptional coactivators p52 and p75-lens epithelium-derived growth factor (LEDGF). Cancer Res. 2000;60:6227-6229.

Alizadeh et al. (2000) “Distinct types of diffuse large B-cell lymphoma identified by gene expression profiling. “Nature” 403:503-511.

Andrews R G, Singer J W, Bernstein I-D. Monoclonal antibody 12-8 recognizes a 115-kd molecule present on both unipotent and multipotent hematopoictic colony-forming cells and their precursors. Blood. 1986; 67:842-845.

Andrews R G, Bryant E M, Bartelmez S H, et al. CD34+ marrow cells, devoid of T and B lymphocytes, reconstitute stable lymphopoiesis and myelopoiesis in lethally irradiated allogeneic baboons. Blood. 1992;80:1693-1701.

Brandt J E, Galy A H, Luens K M et al. Bone marrow repopulation by human marrow stem cells after long-term expansion culture on a porcine endothelial cell line. Exp. Hematol. 1998; 26(10):950-61.

Brandt J E, Bartholomew A M, Fortman J D, et al. Ex vivo expansion of autologous bone marrow CD34+ cells with porcine microvascular endothelial cells results in a graft capable of rescuing lethally irradiated baboons. Blood. 1999;94:106-113.

Brown D, Kogan S, Lagasse E, et al. A PMLRARalpha transgene initiates murine acute promyelocytic leukemia. Proc Natl Acad Sci U S A. 1997;94:2551-2556.

de The H, Lavau C, Marchio A, et al. The PML-RAR alpha fusion mRNA generated by the t(15; 17) translocation in acute promyclocytic leukemia encodes a functionally altered RAR. Cell. 1991;66:675-684.

Golub T, Barker G, Bohlander S, et al. Fusion of the TEL gene on 12p13 to the AML1 gene on 21q22 in acute lymphoblastic leukemia. Proc Natl Acad Sci U S A. 1995;92:4917-4921.

Gomes I, Sharma T, Mahmud N, et al. Highly abundant genes in the transcriptosome of human and baboon CD34 antigen-positive bone marrow cells. Blood. 2001;98:93-99.

Goodell M A, Rosenzweig M, Kim H, et al. Dye efflux studies suggest that hematopoietic stem cells expressing low or undetectable levels of CD34 antigen exist in multiple species. Nat. Med. 1997;3:1337-1345.

Hamdi H, Nishio H, Zielinski R, Dugaiczyk A. Origin and phylogenetic distribution of Alu DNA repeats: irreversible events in the evolution of primates. J. Mol. Biol. 1999;289: 861-871.

Hamdi H K, Nishio H, Tavis J, Zielinski R, Dugaiczyk A. Alu-mediated phylogenetic novelties in gene regulation and development. J. Mol. Biol. 2000;299: 931-939.

Kroon E, Thorsteinsdottir U, Mayotte N, Nakamura T, Sauvageau G. NUP 98-HOXA9 expression in hemopoietic stem cells induces chronic and acute myeloid leukemias in mice. EMBO J. 2001;20:350-361.

Kulkami S, Reiter A, Smedley D, Goldman J, Cross N. The genomic structure of ZNF198 and location of breakpoints in the t(8;13) myeloproliferative syndrome. Genomics. 1999;55:118-121.

Lawrence H, Sauvageau G, Ahmadi N, et al. Stage- and lineage-specific expression of the HOXA10 homeobox gene in normal and leukemic hematopoietic cells. Exp Hematol. 1995;23:1160-1166.

Lee M, Temizer D, Clifford J, Quertermous T. Cloning of the GATA-binding protein that regulates endothelin- 1 gene expression in endothelial cells. J Biol Chem. 1991;266:16188-16192.

Liao D, Pavelitz T, Weiner A M. Characterization of a novel class of interspersed LTR elements in primate genomes: structure, genomic distribution, and evolution. J. Mol. Evol. 1998; 46: 649-660.

Link H, Arseniev L, Bahre O, Kadar J G, Diedrich H, Poliwoda H. Transplantation of allogeneic CD34+ blood cells. Blood. 1996;87:4903-4909.

Look A. Oncogenic transcription factors in the human acute leukemias. Science. 1997;278:1059-1064.

McNeil S, Zeng C, Harrington K, et al. The t(8;21) chromosomal translocation in acute myelogenous leukemia modifies intranuclear targeting of the AML1/CBFalpha2 transcription factor. Proc Natl Acad Sci U S A. 1999;96:14882-14887.

Nachtman R G, Abdullah J M, Jurecic R. Cloning and functional characterization of novel genes preferentially expressed in hematopoietic cells [Abstract]. 29th Annual Meeting of the International Society for Experimental Hematology, Tampa, Fla.: 2000; 28:108.

Orkin S H. Transcription Factors and Hematopoietic Development. J. Biol. Chem. 1995:4955-4958.

Pabst T, Mueller B, Zhang P, et al. Dominant-negative mutations of CEBPA, encoding CCAAT/enhancer binding protein-alpha (C/EBPalpha), in acute myeloid leukemia. Nat Genet. 2001;27:263-270.

Phillips R L, Ernst R E, Brunk B, et al. The genetic program of hematopoictic stem cells. Science. 2000;288:1635-1640.

Pierelli L, Scambia G, Bonanno G, et al. CD34+/CD105+ cells are enriched in primitive circulating progenitors residing in the GO phase of the cell cycle and contain all bone marrow and cord blood CD34+/CD38low/-precursors. Br. J. Haematol. 2000; 108:610-620.

Scott E, Simon M, Anastasi J, Singh H. Requirement of transcription factor PU.1 in the development of multiple hematopoietic lineages. Science. 1994;265:1573-1577.

Skapek S, Jansen D, Wei T, et al. Cloning and characterization of a novel Kruppel-associated box family transcriptional repressor that interacts with the retinoblastoma gene product, RB. J Biol Chem. 2000;275:7212-7223.

Sobek-Klocke I, Disque-Kochem C, Ronsiek M, et al. The human gene ZFP161 on 18p 11.21-pter encodes a putative c-myc repressor and is homologous to murine Zfp161 (Chr 17) and Zfp161-rsl (X Chr). Genomics. 1997;43:156-164.

Tenen D, Hromas R R, Licht J, Yamamishi D, Zhang D. Transciption factors, normal myeloid development, and leukemia. Blood. 1997;90:489-519.

Trezise A E, Godfrey E A, Holmes R S, Beacham I R. Cloning and sequencing of cDNA encoding baboon liver alcohol dehydrogenase: evidence for a common ancestral lineage with the human alcohol dehydrogenase b-subunit and for class I ADH gene duplications predating primate radiation. Proc. Natl. Acad. Sci., U. S. A. 1989;86: 5454-5458.

Ueda T, Yoshino H, Kobayashi K, et al. Hematopoietic repopulating ability of cord blood CD34+ cells in NOD/Shi-scid mice. Stem Cells. 2000;18:204-213.

van Oostveen J, Bijl J, Raaphorst F, Walboomers J, Meijer C. The role of homeobox genes in normal hematopoiesis and hematological malignancies. Leukemia. 1999;13:1675-1690.

Voso M, Burn T, Wulf G, et al. Inhibition of hematopoiesis by competitive binding of transcription factor PU.1. Proc Natl Acad Sci U S A. 1994;91 :7932-7936.

Xiao S, McCarthy J, Aster J, Fletcher J. ZNF198-FGFR1 transforming activity depends on a novel proline-rich ZNF198 oligomerization domain. Blood. 2000;96:699-704.

TABLE 1
Representative sample of vejy highly-abundant named genes in human and
baboon CD34+ cells, by functional category.

| | | | |
|---|---|---|---|
| UniGene | GenBank | | |
| Clusler ID | Accession # | Description | Gene name |

I. Growth Factors/Cytokines

| | | | |
|---|---|---|---|
| Hs. 56023 | AA262988 | Brain-derived neurotrophic | BDNF |
| | | factor | |
| Hs. 180577 | AA496452 | Granulin | GRN |
| Hs. 251664 | N54596 | Insulin-like growth factor 2 | IGF2 |
| Hs. 82045 | AA968896 | Midkine | MDK |
| Hs. 118787 | AA633901 | Transforming growth factor, | TGFBI |
| | | beta-induced | |

II. Cell Surface/Receptois

| | | | |
|---|---|---|---|
| Hs. 85258 | AA443649 | CD8 antigen, alpha | CD8A |
| | | polypeptide | |
| Hs. 75626 | AA136359 | CD58 antigen | CD58 |
| Hs. 75564 | AA456183 | CD151 antigen | CD151 |
| Hs. 2175 | AA443000 | Colony stimulating factor | CSF3R |
| | | 3 precursor receptor | |
| Hs. 110849 | AA098896 | Estrogen-related receptor | BSRRA |
| | | alpha | |
| Hs. 89650 | R68805 | Integral transmembrane | ITM1 |
| | | protein 1 | |
| Hs. 1724 | AA903183 | Interleukin 2 receptor, alpha | IL2RA |
| Hs. 172689 | W44701 | Interleukin 3 receptor, alpha | IL3RA |
| Hs. 47860 | N63949 | Neurotrophic tyrosine kinase, | NTRK2 |
| | | receptor, type 2 | |
| Hs. 82028 | AA487034 | Transforming growth factor, | TGFBR2 |
| | | beta receptor II | |

III. Intracellular signalling molecules

| | | | |
|---|---|---|---|
| Hs. 166154 | AA463972 | jagged 2 | JAG2 |
| Hs. 86859 | 1153703 | Growth factor receptor-bound | GRB7 |
| | protein 7 | | |
| Hs. 78793 | AA447574 | Protein kinase C, zeta | PRKCZ |
| Hs. 62402 | AA890663 | p21/Cdc42/Rac1-activated | PAK1 |
| | | kinase 1 (yeast Ste20-related) | |
| Hs. 75074 | AA455056 | Mitogen-activated protein | MAPKAPK2 |
| | | kinase-activated protein | |
| | | kinase 2 | |
| Hs. 73799 | AA490256 | Guanine nucleotide binding | GNAI3 |
| | | protein, alpha inhibiting | |
| | | activity | |
| Hs. 75217 | AA293050 | Mitogen-activated protein | MAP2K4 |
| | | kinase kinase 4 | |
| Hs. 138860 | AA443506 | Rho GTPase activating | ARHGAP1 |
| | | protein 1 | |

V. Cell cycle proteins

| | | | |
|---|---|---|---|
| Hs. 82906 | AA464698 | Cell division cycle 20, | CDC20 |
| | | S. cercvisiae homolog | |
| Hs. 153752 | AA448659 | Cell division cycle 25B | CDC25B |
| Hs. 172405 | T81764 | Cell division cycle 27 | CDC27 |
| Hs. 77550 | AA459292 | CDC28 protein kinase 1 | CKS1 |

V. Apoptosis/Anti-apoptosis factors

| | | | |
|---|---|---|---|
| Hs. 82890 | AA455281 | Defender against cell death 1 | DAD1 |
| Hs. 227817 | AA459263 | BCL2-related protein A1 | BCL2A1 |

VI. Cytoskeleton/Cell matrix/Adhesion

| | | | |
|---|---|---|---|
| Hs. 183805 | AA464755 | Ankyrin 1, erythrocytic | ANK1 |
| Hs. 171271 | AA442092 | Catenin, beta 1 | CTNNB1 |
| Hs. 75617 | AA430540 | Collagen, type IV, alpha 2 | COL4A2 |
| Hs. 71346 | AA400329 | Neurofilament 3 | NEF3 |
| | | (150 kD medium) | |
| Hs. 78146 | R22412 | Platelet/endothelial cell | PECAM1 |
| | | adhesion molecule | |
| Hs. 75318 | AA180912 | Tubulin, alpha 1 | TUBA1 |

VII. Metabolic proteins

| | | | |
|---|---|---|---|
| Hs. 278399 | AA844818 | Amylase, alpha 2A, | AMY2A |
| | | pancreatic | |
| Hs. 155097 | H23187 | Carbonic anhydrase II | CA2 |
| Hs. 81097 | AA862813 | Cytochrome c oxidase | COX8 |
| | | subunit VIII | |
| Hs. 172690 | AA456900 | Diacylglycerol kinase alpha | DGKA |
| Hs. 944 | AA401111 | Glucose phosphate isomerase | GPI |
| Hs. 2795 | AA489611 | Lactate dehydrogenase A | LDIIA |

VIII. Transcription factors/Activators/Inhibitors

| | | | |
|---|---|---|---|
| Hs. 158195 | AA250730 | Heat shock transcription | HSF2 |
| | | factor 2 | |
| Hs. 22554 | AA252627 | Homeo box B5 | HOXB5 |
| Hs. 153837 | N29376 | Myeloid cell nuclear | MNDA |
| | | differentiation antigen | |
| Hs. 79334 | AA633811 | Nuclear factor, interleukin | NFIL3 |
| | | 3 regulated | |
| Hs. 74002 | AA495962 | Nuelear receptor coactivator 1 | NCOA1 |
| Hs. 192861 | N71628 | Spi-B transcription factor | SPI-B |
| Hs. 3005 | AA284693 | Transcription factor AP-4 | TFAP4 |

Genes highlighted in bold are known to be expressed in hematopoietic tissues
GenBank accession # specifies a cDNA from a specific IMAGE clone spotted on the GeneFilter membrane

TABLE 2
Selection of very highly-abundant ESTs and partially characterized cDNAs
in human and baboon CD34+ Cells.

| UniGene | Genbank | | Gene |
|---|---|---|---|
| Cluster ID | accession # | Description | Name |
| Hs. 155545 | AA423944 | 37 kDa leucine-rich repeat | P37NB |
| | | (LRR) protein | |
| Hs. 42322 | AA682795 | A kinase (PRKA) anchor | AKAP2 |
| | | protein 2 | |
| Hs. 155586 | N90281 | B7 protein | B7 |
| Hs. 118724 | AA406285 | DR1-associated protein 1 | DRAP1 |
| | | (negative cofactor 2 alpha) | |
| Hs. 183738 | AA486435 | FERM, RhoGEF (ARHGEF) | FARP1 |
| | | and pleckstrin domain | |
| | | protein 1 (chondrocyte-de | |
| Hs. 9914 | AA701860 | follistatin | FST |
| Hs. 147189 | R01638 | HYA22 protein | HYA22 |
| Hs. 23119 | AA455272 | ITBA1 gene | ITBA1 |
| Hs. 20149 | AA425755 | leukemia associated gene 1 | LEU1 |
| Hs. 118796 | AA872001 | Annexin A6 | ANX6 |
| Hs. 102948 | AA127096 | enigma (LIM domain protein) | ENIGMA |
| Hs. 41007 | AA147980 | HSPC158 protein | HSPC158 |
| Hs. 89650 | R68805 | integral membrane protein 1 | ITM1 |
| Hs. 69855 | AA504682 | NRAS-related gene | DIS155E |
| Hs. 172589 | AA485992 | nuclear phosphoprotein | PWP1 |
| | | similar to S. cerevisiae PWP1 | |
| Hs. 2815 | N63968 | POU domain, class 6, | POU6F1 |
| | | transcription factor 1 | |
| Hs. 59545 | AA195036 | ring finger protein 15 | RNF15 |
| Hs. 172052 | AA732873 | serine/threonine kinase 18 | STK18 |
| Hs. 444 | H87351 | serine/threonine kinase 19 | STK19 |
| Hs. 98874 | AA436479 | similar to proline-rich | LOC54518 |
| | | protein 48 | |
| Hs. 151689 | AA043458 | zinc finger protein 137 | ZNF137 |
| | | (clone pHZ-30) | |
| Hs. 169832 | AA120779 | zinc finger protein 42 | ZNF42 |
| | | (myeloid-specific retinoic | |
| | | acid-responsive) | |
| Hs. 104746 | AA406206 | ESTs, Highly similar to NBL4 | |
| | | PROTEIN [M. musculus] | |
| Hs. 58643 | AA490900 | ESTs, Highly similar to | |
| | | JAK3B [H. sapiens] | |
| Hs. 42733 | W85875 | ESTs. Weakly similar to BC-2 | |
| | | protein [H. sapiens] | |
| Hs. 90020 | AA626316 | ESTs, Weakly similar to | |
| | | KINESIN LIGHT CHAIN | |
| | | [H. sapiens] | |
| Hs. 118739 | AA521439 | ESTs, Weakly similar to | |
| | | phosphoinositide 3-kinase | |
| | | [H. sapiens] | |
| Hs. 84640 | W93317 | ESTs, Weakly similar to | |
| | | proline-rich protein MP3 | |
| | | [M. musculus] | |
| Hs. 24956 | AA454654 | ESTs, Weakly similar to SH3 | |
| | | domain-binding protein | |
| | | SNP70 [H. sapiensυ | |
| Hs: 36779 | H53499 | ESTs, Weakly similar to | |
| | | Zn-finger-like protein | |
| | | [H. sapiens] | |

GenBank accession # specifies a cDNA from a specific IMAGE clone spotted on the GeneFilter membrane

TABLE 3
Comparison of expression level of apparent species-specific genes
by semi-quantitative RT-PCR.

| | | | | Hu/Bab | |
|---|---|---|---|---|---|
| | | | Hu/Bab | Intensity | |
| | | | Intensity | Ratio | |
| Specificity | Unigene | Primer | Ratio | (by | Gene |
| (by GFs) | Cluster ID | Pair | (by GFs) | RT-PCR) | Name |
| Human | Hs.1817 | R05886 | 16.3 | 3.6 | MPO |
| Human | Hs.13818 | R85439 | 6.9 | 1.5 | ESTs |
| Human | Hs.47956 | N55359 | 4.9 | * | ESTs |
| Human | Hs.43708 | N25920 | 3.7 | −1.9 | EST |
| Human | Hs.215595 | AA487912 | 3.2 | 5.4 | GNB1 |
| Baboon | Hs.118409 | AA676327 | −21.5 | 1.8 | ESTs |
| Baboon | Hs.107308 | R82595 | −19.3 | 1.2 | cDNA |
| Baboon | Hs.114593 | N74363 | −9.2 | * | ESTs |

Primer pairs were named after the GenBank Accession number specifying a cDNA from a specific IMAGE clone spotted on GeneFilter membrane GF, GeneFilters; MPO, myetoperoxidase; GNB1, Guanine nucteotide binding protein (G protein), beta polypeptide 1; cDNA, Homo sapiens uncharacterized gene.
*indicates no expression in either species. Negative intensity ratio indicates higher expression in baboon than in human.

TABLE 4A
Potential human transcriptional regulators selected from the human CD34+ transcriptosome database.

| | | | | | | |
|---|---|---|---|---|---|---|
| Unigene | Gene | | | | Abund- | Character- |
| Cluster | Name | Gene Description | Band | FB | ance | ization |

TRANSCRIPTION

| | | | | | | |
|---|---|---|---|---|---|---|
| Hs.321677 | STAT3 | signal transducer and activator of transcription 3 (acute- | 17q21 | 437.5 | VH | W |
| | | phase response factor) | | | | |
| Hs.78881 | MEF2B | MADS box transcription enhancer factor 2, polypeptide B | 19p12 | 432.8 | VH | W |
| | | (myocyte enhancer factor 2B) | | | | |
| Hs.96055 | E2F1 | E2F transcription factor I | 20q11.2 | 224.9 | VH | W |
| *Hs.192861 | SPIB | Spi-D transcription factor (Spi-1/PU.1 related) | 19q13.3-q13.4 | 207.0 | VH | W |
| Hs.22302 | GTF3C4 | general transcription factor IIIC, polypeptide 4 (90 kD) | 9 | 200.8 | VH | W |
| Hs.74861 | PC4 | activated RNA polymerase II transcription cofactor 4 | 8 | 151.9 | VH | W |
| Hs.3005 | TFAP4 | transcription factor AP-4 (activating enhancer-binding | 16p13 | 146.9 | VH | W |
| | | protein 4) | | | | |
| Hs.79353 | TFDP1 | transcription factor Dp-1 | 13q34 | 144.2 | VH | W |
| Hs.2815 | POU6F1 | POU domain, class 6, transcription factor 1 | 12 | 138.6 | VH | PC |
| Hs.19131 | TFDP2 | transcription factor Dp-2 (E2F dimerization partner 2) | 3q23 | 118.5 | VH | W |
| Hs.158195 | HSF2 | heat shock transcription factor 2 | 6pter-p25.1 | 110.9 | VH | W |
| Hs.239720 | CNOT2 | CCR4-NOT transcription complex, subunit 2 | 12 | 99.4 | H | PC |
| Hs.93748 | | Homo sapiens clone moderately similar to Transcription | N/A | 81.7 | H | N |
| | | Factor BTF3 | | | | |
| Hs.110103 | RRN3 | RNA polymerase I transcription factor RRN3 | 16p12 | 68.9 | H | W |
| Hs.334334 | TFAP2A | transcription factor AP-2 alpha (activating enhancer- | 6p24 | 63.8 | H | W |
| | | binding protein 2 alpha) | | | | |
| Hs.80598 | TCEA2 | transcription elongation factor A (SII), 2 | N/A | 59.4 | H | W |
| Hs.108106 | ICBP90 | transcription factor | 19p13.3 | 54.0 | H | PC |
| Hs.154970 | TFCP2 | transcription factor CP2 | 12q13 | 48.0 | H | W |
| Hs.294101 | PBX3 | pre-B-cell leukemia transcription factor 3 | 9q33-q34 | 43.1 | H | PC |
| *Hs.274184 | TFE3 | transcription factor binding to IGHM enhancer 3 | Xp11.22 | 40.9 | H | W |
| Hs.108371 | E2F4 | E2F transcription factor 4, p107/p130-binding | 16q21-q22 | 40.4 | H | W |
| Hs.95243 | TCEAL1 | transcription elongation factor A (SII)-like I | Xq22.1 | 38.8 | H | W |
| Hs.1706 | ISGF3G | interferon-stimulated transcription factor 3, gamma (48 kD) | 14q11.2 | 37.9 | H | W |
| Hs.9754 | ATF5 | activating transcription factor 5 | 19q13.3 | 34.6 | H | W |
| Hs.244613 | STAT5B | signal transducer and activator of transcription 5B | 17q11.2 | 33.6 | H | W |
| Hs.169294 | TCF7 | transcription factor 7 (T-cell specific, HMG-box) | 5q31.1 | 31.7 | H | W |
| Hs.249184 | TCF19 | transcription factor 19 (SC1) | 6p21.3 | 29.5 | H | PC |
| Hs.7647 | MAZ | MYC-associated zinc finger protein (purine-binding | 16p11.2 | 27.3 | H | W |
| | | transcription factor) | | | | |
| Hs.29417 | ZF | HCF-binding transcription factor Zhangfei | 11q14 | 24.0 | I | PC |
| Hs.155313 | DATF1 | death associated transcription factor 1 | 20 | 22.3 | I | PC |
| Hs.182280 | MEF2A | MADS box transcription enhancer factor 2, polypeptide A | 15q26 | 21.5 | I | W |
| | | (myocyte enhancer factor 2A) | | | | |
| Hs.26703 | CNOT8 | CCR4-NOT transcription complex, subunit 8 | 5q31-q33 | 21.4 | I | PC |
| Hs.279818 | AF093680 | similar to mouse Glt3 or D. malanogaster transcription | 16q13-q21 | 21.3 | I | W |
| | | factor IIB | | | | |
| Hs.1189 | E2F3 | E2F transcription factor 3 | 6p22 | 21.0 | I | W |
| Hs.68257 | GTF2F1 | general transcription factor IIF, polypeptide 1 (74 kD | 19p13.3 | 19.0 | I | W |
| | | subunit) | | | | |
| Hs.197540 | HIF1A | hypoxia-inducible factor 1, alpha subunit (basic helix-loop- | 14q21-q24 | 16.4 | I | W |
| | | helix transcription factor) | | | | |
| *Hs.211588 | POU4F1 | POU domain, class 4, transcription factor 1 | 13q21.1-q22 | 15.9 | I | W |
| *Hs.155321 | SRF | serum response factor (c-fos serum response element- | 6pter-p24.1 | 15.1 | I | W |
| | | binding transcription factor) | | | | |
| Hs.103989 | NCYM | DNA-binding transcriptional activator | 2p24.1 | 14.9 | I | W |
| Hs.326198 | TCF4 | transcription factor 4 | 18q21.1 | 14.7 | I | W |
| Hs.75133 | TCF6L1 | transcription factor 6-like 1 (mitochondrial transcription | 7pter-cen | 14.2 | I | W |
| | | factor 1-like) | | | | |
| Hs.101025 | BTF3 | basic transcription factor 3 | 5 | 14.1 | I | W |
| Hs.166096 | ELF3 | E74-like factor 3 (ets domain transcription factor, epithelial- | 1q32.2 | 13.8 | I | W |
| | | specific) | | | | |
| Hs.797 | NFYA | nuclear transcription factor Y, alpha | 6p21.3 | 13.7 | I | W |
| Hs.129914 | RUNX1 | runt-related transcription factor 1 (acute myeloid leukemia | 21q22.3 | 13.6 | I | W |
| | | 1; amll oncogene) | | | | |
| Hs.247433 | ATF6 | activating transcription factor 6 | 1q22-q23 | 13.5 | I | W |
| Hs.90304 | GTF2H3 | general transcription factor IIH, polypeptide 3 (34 kD | 12 | 11.9 | I | W |
| | | subunit) | | | | |
| Hs.78061 | TCP21 | transcription factor 21 | 6pter-qter | 11.1 | I | PC |
| Hs.75113 | GTF3A | general transcription factor IIIA | 13q12.3-q13.1 | 10.0 | I | W |
| Hs.93728 | PBX2 | pre-B-cell leukemia transcription factor 2 | 6p21.3 | 9.8 | L | W |
| Hs.16697 | DR1 | down-regulator of transcription 1, TBP-binding (negative | 1p22.1 | 9.8 | L | W |
| | | cofactor 2) | | | | |
| Hs.182237 | POU2F1 | POU domain, class 2, transcription factor 1 | 1q22-q23 | 9.7 | L | W |
| Hs.765 | GATA1 | GATA-binding protein 1 (globin transcription factor 1) | Xp11.23 | 9.0 | L | W |
| Hs.101842 | ATBF1 | AT-binding transcription factor 1 | 16q22.3-q23.1 | 8.7 | L | W |
| Hs.211581 | MTF1 | metal-regulatory transcription factor 1 | 1p33 | 8.2 | L | W |
| Hs.173854 | PAXIPIL | PAX transcription activation domain interacting protein 1 | 7q36 | 8.2 | L | N |
| | | like | | | | |
| Hs.197764 | TITF1 | thyroid transcription factor 1 | 14q13 | 8.0 | L | W |
| Hs.2982 | SP4 | Sp4 transcription factor | 7p15 | 7.6 | L | W |
| Hs.59506 | DMRT2 | doublesex and mab-3 related transcription factor 2 | 9p24.3 | 7.0 | L | PC |
| Hs.21486 | STAT1 | signal transducer and activator of transcription 1, 91 kD | 2q32.2 | 6.9 | L | W |
| Hs.278589 | GTP2I | general transcription factor II, i | 7q11.23 | 6.6 | L | W |
| Hs.121895 | RUNX2 | runt-related transcription factor 2 | 6p21 | 6.6 | L | W |
| Hs.460 | ATF3 | activating transcription factor 3 | 1 | 6.3 | L | W |
| Hs.268115 | | ESTs, Weakly similar to T08599 probable transcription | N/A | 6.3 | L | N |
| | | factor CA150 [H. sapiens] | | | | |
| Hs.171626 | TCEBIL | transcription elongation factor B (SIII), polypeptide 1-like | 5q31 | 6.1 | L | W |
| *Hs.1101 | POU2F2 | POU domain, class 2, transcription factor 2 | 19 | 6.1 | L | W |
| Hs.54780 | TTF1 | transcription termination factor, RNA polymerase 1 | 9 | 5.9 | L | W |
| *Hs.89781 | UBTF | upstream binding transcription factor, RNA polymerase 1 | 17q21.3 | 5.8 | L | W |
| *Hs.14963 | FACTP140 | chromatin-specific transcription elongation factor, 140 kDa | 14 | 5.8 | L | W |
| | | subunit | | | | |
| Hs.198166 | ATF2 | activating transcription factor 2 | 2q32 | 5.7 | L | W |
| Hs.30824 | LZTFL1 | leucine zipper transcription factor-like 1 | 3p21.3 | 5.5 | L | N |
| Hs.108300 | CNOT3 | CCR4-NOT transcription complex, subunit 3 | 19q13.4 | 5.3 | L | W |
| Hs.166017 | MITF | microphthalmia-associated transcription factor | 3p14.1-p12.3 | 5.0 | L | W |
| Hs.181243 | ATF4 | activating transcription factor 4 (tax-responsive enhancer | 22q13.1 | 4.9 | L | W |
| | | element B67) | | | | |
| Hs.2331 | E2F5 | E2F transcription factor 5, p130-binding | 8p22 | 4.9 | L | W |
| Hs.173638 | TCF7L2 | transcription factor 7-like 2 (T-cell specific, HMG-box) | 10q25.3 | 4.9 | L | W |
| Hs.24572 | | ESTs, Weakly similar to TC17_HUMAN | N/A | 4.8 | L | N |
| | | TRANSCRIPTION FACTOR 17 [H. sapiens] | | | | |
| Hs.191356 | GTF2H2 | general transcription factor IIH, polypeptide 2 (44 kD | 5q12.2-q13.3 | 4.6 | L | W |
| | | subunit) | | | | |
| *Hs.78869 | TCEA1 | transcription elongation factor A (SII), 1 | 3p22-p21.3 | 4.6 | L | W |
| Hs.170019 | RUNX3 | runt-related transcription factor 3 | 1p36 | 4.4 | L | W |
| Hs.154276 | BACH1 | BTB and CNC homology 1, basic leucine zipper | 21q22.11 | 4.4 | L | W |
| | | transcription factor 1 | | | | |
| Hs.184771 | NFIC | nuclear factor I/C (CCAAT-binding transcription factor) | 19p13.3 | 4.4 | L | W |
| Hs.89578 | GTF2H1 | general transcription factor IIH, polypeptide 1 (62 kD | 11p15.1-p14 | 4.3 | L | W |
| | | subunit) | | | | |
| Hs.227630 | REST | RE1-silencing transcription factor | 4q12-q13.3 | 4.3 | L | W |
| Hs.21704 | TCF12 | transcription factor 12 (HTF4, helix-loop-helix | 15q21 | 4.3 | L | PC |
| | | transcription factors 4) | | | | |
| Hs.226318 | CNOT7 | CCR4-NOT transcription complex, subunit 7 | 8p22-p21.3 | 4.1 | L | PC |
| *Hs.13063 | CA150 | transcription factor CA150 | 5q31 | 4.1 | L | W |
| Hs.150557 | BTEB1 | basic transcription element binding protein 1 | 9q13 | 4.1 | L | W |
| Hs.84928 | NFYB | nuclear transcription factor Y, beta | 12q22-q23 | 4.1 | L | W |
| Hs.35841 | NFIX | nuclear factor I/X (CCAAT-binding transcription factor) | 19p13.3 | 4.0 | L | PC |
| Hs.151139 | ELF4 | E74-like factor 4 (ets domain transcription factor) | Xq26 | 3.9 | L | W |
| Hs.78995 | MEF2C | MADS box transcription enhancer factor 2, polypeptide C | 5q14 | 3.9 | L | W |
| | | (myocyte enhancer factor 2C) | | | | |
| Hs.97996 | MTERF | transcription termination factor, mitochondrial | 7q21-q22 | 3.9 | L | W |
| Hs.119018 | NRF | transcription factor NRF | Xp21.1-q25 | 3.8 | L | W |
| Hs.100932 | TCF17 | transcription factor 17 | 5q35.3 | 3.5 | L | PC |
| Hs.92282 | PITX2 | paired-like homeodomain transcription factor 2 | 4q25-q27 | 3.4 | L | PC |
| Hs.169853 | TCF2 | transcription factor 2, hepatic; LF-B3, variant hepatic- | 17cen-q21.3 | 3.4 | L | W |
| | | nuclear factor | | | | |
| Hs.181015 | STAT6 | signal transducer and activator of transcription 6, | 12q13 | 3.3 | L | W |
| | | interleukin-4 induced | | | | |
| Hs.97624 | HSF2BP | heat shock transcription factor 2 binding protein | 21q22.3 | 3.2 | L | PC |
| Hs.171185 | P38IP | transcription factor (p38 interacting protein) | 13q12.2- | 3.2 | L | N |
| | | | 13q14.2 | | | |
| Hs.184693 | TCEB1 | transcription elongation factor B (SIII), polypeptide 1 | 8 | 3.1 | L | W |
| | | (15 kD, elongin C) | | | | |
| Hs.20423 | CNOT4 | CCR4-NOT transcription complex, subunit 4 | 7q22-qter | 3.1 | L | PC |
| Hs.76362 | GTF2A2 | general transcription factor IIA, 2 (12 kD subunit) | 15q11.2 | 3.1 | L | W |
| *Hs.2430 | TCFL1 | transcription factor-like 1 | 1q21 | 3.0 | L | PC |
| Hs.166 | SREBF1 | sterol regulatory element binding transcription factor 1 | 17p11.2 | 3.0 | L | W |
| ZINC | | | | | | |
| *Hs.194688 | BAZ1B | bromodomain adjacent to zinc finger domain, 1B | 7q11.23 | 439.3 | VH | PC |
| Hs.150390 | ZNF262 | zinc finger protein 262 | 1p32-p34 | 369.5 | VH | N |
| Hs.1148 | ZFP | zinc finger protein | 3p22.3-p21.1 | 244.4 | VH | N |
| Hs.301637 | ZNF258 | zinc finger protein 258 | 14q12 | 231.9 | VH | N |
| Hs.6557 | ZNF161 | zinc finger protein 161 | 3q26.2 | 139.7 | VH | PC |
| Hs.108139 | ZNF212 | zinc finger protein 212 | 7q36.1 | 118.5 | VH | N |
| Hs.169832 | ZNF42 | zinc finger protein 42 (myeloid-specific retinoic acid- | 19q13.2-q13.4 | 117.3 | VH | W |
| | | responsive) | | | | |
| Hs.277401 | BAZ2A | bromodomain adjacent to zinc finger domain, 2A | 12q24.3-qter | 108.3 | VH | N |
| Hs.151689 | ZNF137 | zinc finger protein 137 (clone pHZ-30) | 19q13.4 | 107.3 | VH | N |
| Hs.96448 | ZNP193 | zinc finger protein 193 | 6p21.3 | 105.2 | VH | N |
| *Hs.58167 | ZNF282 | zinc finger protein 282 | 7q35-q36 | 98.4 | H | PC |
| Hs.3057 | ZNF74 | zinc finger protein 74 (Cos52) | 22q11.21 | 88.8 | H | PC |
| Hs.70617 | ZNF33A | zinc finger protein 33a (KOX 31) | 10p11.2 | 78.6 | H | N |
| Hs.165983 | FLJ22504 | hypothetical C2H2 zinc finger protein FLJ22504 | 20q11.21- | 62.6 | H | N |
| | | | q13.12 | | | |
| Hs.27801 | ZNF278 | zinc finger protein 278 | 22q12.2 | 57.8 | H | W |
| Hs.194718 | ZNF265 | zinc finger protein 265 | 1p31 | 57.2 | H | PC |
| Hs.142634 | AF020591 | zinc finger protein | 19 | 50.6 | H | N |
| Hs.183593 | ZNF24 | zinc finger protein 24 (KOX 17) | 18q12 | 46.1 | H | PC |
| Hs.180677 | ZNF162 | zinc finger protein 162 | 11q13 | 46.0 | H | W |
| Hs.288773 | ZNF294 | zinc finger protein 294 | 21q22.11 | 40.8 | H | N |
| Hs.22879 | LOC51193 | zinc finger protein ANC_2H01 | 3q25.1-q25.33 | 39.5 | H | N |
| Hs.13128 | ZNF205 | zinc finger protein 205 | 16p13.3 | 35.9 | H | N |
| Hs.182528 | ZNF263 | zinc finger protein 263 | 16 | 35.4 | H | PC |
| Hs.119014 | ZNF175 | zinc finger protein 175 | 19q13.4 | 29.9 | H | N |
| Hs.117077 | ZNF264 | zinc finger protein 264 | 19q13.4 | 27.4 | H | N |
| *Hs.82210 | ZNF220 | zinc finger protein 220 | 8p11 | 24.5 | I | PC |
| Hs.12940 | ZHX1 | zine-fingers and homeoboxes 1 | 8q | 24.0 | I | PC |
| Hs.8383 | BAZ2B | bromnodomain adjacent to zinc finger domain, 2B | 2q23-q24 | 22.5 | I | N |
| Hs.288658 | ZNF35 | zinc finger protein 35 (clone HF10) | 3p22-p21 | 22.1 | I | PC |
| Hs.132390 | ZNF36 | zinc finger protein 36 (KOX 18) | 7q21.3-q22 | 21.4 | I | N |
| Hs.7137 | LOC57862 | clones 23667 and 23775 zinc finger protein | 14q24.3 | 20.3 | H | N |
| Hs.110839 | ZFP95 | zinc finger protein homologous to Zfp95 in mouse | 7q22 | 15.5 | I | N |
| Hs.10590 | ZNF313 | zinc finger protein 313 | 20q11.21- | 15.1 | I | N |
| | | | q11.23 | | | |
| Hs.86356 | | EST, Weakly similar to Z117_HUMAN ZINC FINGER | N/A | 14.3 | I | N |
| | | PROTEIN 117 [H. sapiens] | | | | |
| Hs.48589 | ZNF228 | zinc finger protein 228 | 19q13.2 | 14.1 | I | N |
| Hs.50216 | ZFD25 | zinc finger protein (ZFD25) | 7q11.2 | 12.5 | I | N |
| Hs.301819 | ZNF146 | zinc finger protein 146 | 19q13.1 | 10.9 | I | PC |
| Hs.74107 | ZNF43 | zinc finger protein 43 (HTF6) | 19p13.1-p12 | 10.6 | I | PC |
| Hs.33532 | ZNF151 | zinc finger protein 151 (pHZ-67) | 1p36.2-p36.1 | 10.4 | I | W |
| Hs.20047 | ZNFN2A1 | zinc finger protein, subfamily 2A (FYVE domain | 14q22-q24 | 10.4 | I | N |
| | | containing), 1 | | | | |
| Hs.289104 | ABP/ZF | Alu-binding protein with zinc finger domain | 7 | 10.3 | I | N |
| Hs.57419 | CTCF | CCCTC-binding factor (zinc finger protein) | 16q21-q22.3 | 9.9 | L | W |
| *Hs.2110 | ZNF9 | zinc finger protein 9 (a cellular retroviral nucleic acid | 3q13.3-q24 | 9.8 | L | PC |
| | | binding protein) | | | | |
| Hs.93005 | SLUG | slug (chicken homolog), zinc finger protein | 8q11 | 9.8 | L | W |
| *Hs.158174 | ZNF184 | zinc finger protein 184 (Knippel-like) | 6p21.3 | 9.8 | L | N |
| Hs.59757 | ZNF281 | zinc finger protein 281 | 1q32.1 | 9.7 | L | PC |
| *Hs.15220 | ZFP106 | zinc finger protein 106 | 15 | 9.6 | L | N |
| Hs.237786 | ZNF187 | zinc finger protein 187 | 6p22 | 8.9 | L | PC |
| Hs.62112 | ZNF207 | zinc finger protein 207 | 17 | 8.8 | L | N |
| Hs.270435 | FLJ12985 | hypothetical protein FLJ12985, HUMAN ZINC FINGER | 19q12 | 8.7 | L | N |
| | | PROTIEN 91 | | | | |
| Hs.89732 | ZNF273 | zinc finger protein 273 | N/A | 8.4 | L | N |
| Hs.29159 | ZNF75 | zinc finger protein 75 (D8C6) | Xq26 | 8.0 | L | N |
| Hs.24125 | LOC51780 | putative zinc finger protein | 5q31 | 7.8 | L | PC |
| Hs.301059 | FLJ12488 | hypothetical protein FLJ12488, moderately HUMAN ZINC | N/A | 7.5 | L | N |
| | | FINGER PROTEIN 93 | | | | |
| Hs.19585 | SZF1 | KRAB-zinc finger protein SZF1-1 | 3p21 | 7.2 | L | PC |
| Hs.154095 | ZNF143 | zinc finger protein 143 (clone pHZ-1) | 11p15.4 | 7.1 | L | PC |
| *Hs.30503 | | Homo sapiens cDNA FLJ11344 fis, clone | N/A | 7.0 | L | N |
| | | PLACE1010870, moderately similar to ZINC FINGER | | | | |
| | | PROTEIN 91 | | | | |
| Hs.9786 | ZNF275 | zinc finger protein 275 | N/A | 6.7 | L | N |
| Hs.3053 | ZID | zinc finger protein with interaction domain | 9q33.1-q33.3 | 6.3 | L | PC |
| Hs.20631 | PEGASUS | zinc finger protein, subfamily 1A, 5 (Pegasus) | 10q26 | 6.0 | L | PC |
| Hs.20082 | ZNF3 | zinc finger protein 3 (A8-51) | 5 | 5.9 | L | N |
| *Hs.108642 | ZNF22 | zinc finger protein 22 (KOX 15) | 10qt11 | 5.5 | L | N |
| Hs.78743 | ZNF131 | zinc finger protein 131 (clone pHZ-10) | 5p12-p11 | 5.5 | L | N |
| Hs.29222 | ZNF76 | zinc finger protein 76 (expressed in testis) | 6p21.3-p21.2 | 5.4 | L | PC |
| *Hs.287331 | ZNF286 | zinc finger protein ZNF286 | 17p11.2 | 5.3 | L | N |
| *Hs.69997 | ZNF238 | zinc finger protein 238 | 1q44-qter | 5.3 | L | PC |
| Hs.109526 | ZNF198 | zinc finger protein 198 | 13q11-q12 | 5.3 | L | PC |
| Hs.85505 | | ESTs, Weakly similar to ZF37_HUMAN ZINC FINGER | N/A | 5.3 | L | N |
| | | PROTEIN ZFP-37 [H sapiens] | | | | |
| Hs.48029 | SNAI1 | snail 1 (drosophila homolog), zinc finger protein | 20q13.1-q13.2 | 5.1 | L | PC |
| Hs.172979 | ZNF177 | zinc finger protein 177 | 19pter-19p13.3 | 4.8 | L | PC |
| Hs.155204 | ZNF174 | zinc finger protein 174 | 16p13.3 | 4.8 | L | PC |
| Hs.86371 | ZNF254 | zinc finger protein 254 | 19p13.12- | 4.8 | L | N |
| | | | p13.11 | | | |
| Hs.180248 | ZNF124 | zinc finger protein 124 (HZF-16) | 1q44 | 4.6 | L | PC |
| Hs.279914 | ZNF232 | zinc finger protein 232 | 17p13-p12 | 4.6 | L | PC |
| Hs.15110 | ZNF211 | zinc finger protein 211 | 19q13.4 | 4.3 | L | N |
| Hs.55481 | ZNF165 | zinc finger protein 165 | 6p21.3 | 4.3 | L | N |
| Hs.33268 | | Homo sapiens weakly similar to ZINC FINGER PROTEIN | N/A | 4.2 | L | N |
| | | 84 | | | | |
| Hs.197219 | ZNF14 | zinc finger protein 14 (KOX 6) | 19p13.3-p13.2 | 4.2 | L | N |
| Hs.296365 | ZF5128 | zinc finger protein | 19 | 4.1 | L | N |
| Hs.22182 | ZNF23 | zinc finger protein 23 (KOX 16) | 16q22 | 4.1 | L | N |
| Hs.183291 | ZNF268 | zinc finger protein 268 | 5 | 4.0 | L | N |
| Hs.156000 | ZFP161 | zinc finger protein homologous to Zfp161 in mouse | 18pter-p11.2 | 4.0 | L | N |
| Hs.72318 | | Homo sapiens moderately similar to ZINC FINGER | N/A | 4.0 | L | N |
| | | PROTEIN 91 | | | | |
| Hs.64794 | ZNF183 | zinc finger protein 183 (RING finger, C3HC4 type) | Xq25-q26 | 4.0 | L | N |
| *Hs.110956 | ZNF20 | zinc finger protein 20 (KOX 13) | 19p13.3-p13.2 | 4.0 | L | PC |
| Hs.184669 | ZNF144 | zinc finger protein 144 (Mel-18) | 17 | 3.7 | L | W |
| Hs.23476 | CIZI | Cip1-interacting zinc finger protein | 9q34.1 | 3.4 | L | PC |
| Hs.31324 | ZNF155 | zinc finger protein 155 (pHZ-96) | 19q13.2-q13.32 | 3.2 | L | N |
| Hs.23019 | ZNF16 | zinc finger protein 16 (KOX 9) | 8q24 | 3.1 | L | N |
| Hs.88219 | ZNF200 | zinc finger protein 200 | 16p13.3 | 3.1 | L | N |

ACTIVATOR

| | | | | | | |
|---|---|---|---|---|---|---|
| Hs.146847 | TANK | TRAF family member-associated NFKB activator | 2q24-q31 | 84.0 | H | W |
| Hs.40403 | CITED1 | Cbp/p300-interacting transactivator, with Glu/Asp-rich | Xq13.1 | 37.8 | H | W |
| | | carboxy-terminal domain, 1 | | | | |
| Hs.198468 | PPARGC1 | peroxisome proliferative activated receptor, gamma, | 4p15.1 | 13.7 | I | W |
| | | coactivator 1 | | | | |
| Hs.82071 | CITED2 | Cbp/p300-interacting transactivator, with Glu/Asp-rich | 6q23.3 | 12.3 | I | W |
| | | carboxy-terminal domain, 2 | | | | |
| Hs.3076 | MHC2TA | MHC class II transactivator | 16p13 | 10.0 | I | W |
| Hs.283689 | ACT | activator of CREM in testis | 6q16.1-q16.3 | 3.9 | L | W |
| Hs.79093 | p100 | EBNA-2 co-activator (100 kD) | 7q31.3 | 3.0 | L | PC |

ENHANCER

| | | | | | | |
|---|---|---|---|---|---|---|
| Hs.83958 | TLE4 | transducin-like enhancer of split 4, homolog of Drosophila | 9 | 721.5 | VH | W |
| | | E(sp1) | | | | |
| Hs226573 | IKBKB | inhibitor of kappa light polypeptide gene enhancer in B- | 8p11.2 | 106.5 | VH | W |
| | | cells, kinase beta | | | | |
| Hs.28935 | TLE1 | transducin-like enhancer of split 1, homolog of Drosophila | 19p13.3 | 58.4 | H | W |
| | | E(sp1) | | | | |
| *Hs.75117 | ILF2 | interleukin enhancer binding factor 2, 45 kD | N/A | 52.4 | H | W |
| Hs.234434 | HEY1 | hairy/enhancer-of-split related with YRPW motif 1 | 8q21 | 29.9 | H | PC |
| Hs.81328 | NFKBIA | nuclear factor of kappa light polypeptide gene enhancer in | 14q13 | 23.2 | I | W |
| | | B-cells inhibitor, alpha | | | | |
| Hs.332173 | TLE2 | transducin-like enhancer of split 2, homolog of Drosophila | 19p13.3 | 18.8 | I | W |
| | | E(sp1) | | | | |
| Hs.99029 | CEBPB | CCAAT/enhancer binding protein (C/EBP), beta | 20q13.1 | 11.1 | I | W |
| Hs.256583 | ILF3 | interlenkin enhancer binding factor 3, 90 kD | 19p13 | 10.0 | I | PC |
| *Hs.83428 | NFKB1 | nuclear factor of kappa light polypeptide gene enhancer in | 4q24 | 9.7 | L | W |
| | | B-cells 1(p105) | | | | |
| Hs.2227 | CEBPG | CCAAT/enhancer binding protein (C/EBP), gamma | 19 | 5.9 | L | W |
| Hs.9731 | NFKBIB | nuclear factor of kappa light polypeptide gene enhancer in | 19q13.1 | 4.6 | L | W |
| | | B-cells inhibibitor, beta | | | | |
| Hs.306 | HIVEP | human immunodeficiency virus type I enhancer-binding | 6p24-p22.3 | 3.8 | L | W |
| | | protein 1 | | | | |
| Hs.76722 | CEBPD | CCAAT/enhancer binding protein (C/EBP), delta | 8p11.2-p11.1 | 3.8 | L | W |

FORKHEAD

| | | | | | | |
|---|---|---|---|---|---|---|
| Hs.44481 | FOXF2 | forkhead box F2 | 6p25.3 | 151.3 | VH | PC |
| Hs.2714 | FOXG1B | forkhead box G1B | 14q12-q13 | 67.9 | H | W |
| Hs.56213 | | ESTs, Highly similar to FXD3_HUMAN FORKHEAD | N/A | 55.0 | H | N |
| | | BOX PROTEIN D3 [H sapiens] | | | | |
| Hs.239 | FOXM1 | forkhead box M1 | 12p13 | 25.9 | H | W |
| Hs.155591 | FOXF1 | forkhead box F1 | 16q24 | 21.8 | I | PC |
| Hs.112968 | FOXE3 | forkhead box E3 | 1p32 | 7.9 | L | PC |
| Hs.284186 | FOXC1 | forkhead box C1 | 6p25 | 6.3 | L | PC |
| *Hs.170133 | FOXO1A | forkliead box O1A (rhabdomyosarcoma) | 13q14.1 | 5.7 | L | PC |
| Hs.96028 | FOXD1 | forkhead box D1 | 5q12-q13 | 4.5 | L | PC |
| Hs.120844 | LOC55810 | FOXJ2 forkhead factor | 12pter-p13.31 | 4.1 | L | PC |
| Hs.93974 | FOXJ1 | forkhead box J1 | 17q22-17q25 | 3.3 | L | PC |
| HELIX | | | | | | |
| Hs.76884 | ID3 | inhibitor of DNA binding 3, dominant negative helix-loop- | 1p36.13-p36.12 | 30.8 | H | W |
| | | helix protein | | | | |
| Hs.198998 | CHUK | conserved helix-loop-helix ubiquitous kinase | 10q24-q25 | 10 | I | W |
| Hs.30956 | NHLH1 | nescient helix loop helix 1 | 1q22 | 8.2 | L | W |
| *Hs.34853 | ID4 | inhibitor of DNA binding 4, dominant negative helix-loop- | 6p22-p21 | 3.7 | L | W |
| | | helix protein | | | | |
| Hs.46296 | NHLH2 | nescient helix loop helix 2 | 1p12-P11 | 3.6 | L | PC |
| Hs.75424 | ID1 | inhibitor of DNA binding 1, dominant negative helix-loop- | 20q11 | 3.3 | L | W |
| | | helix protein | | | | |

HOMEOBOX

| | | | | | | |
|---|---|---|---|---|---|---|
| Hs.55967 | SHOX2 | short stature homeobox 2 | 3q25-q26.1 | 220.3 | VH | PC |
| Hs.125231 | HPX42B | haemopoietic progenitor homeobox | 10q26 | 6.3 | L | PC |
| *Hs.90077 | TGIF | TG-interacting factor (TALE family homeobox) | 18p11.3 | 4.9 | L | W |

LEUCINE

| | | | | | | |
|---|---|---|---|---|---|---|
| Hs.158205 | BLZF1 | basic leucine zipper nuclear factor 1 (JEM-1) | 1q24 | 3.4 | L | PC |
| NON-POU | | | | | | |
| Hs.172207 | NONO | non-POU-domain-containing, octamer-binding | Xq13.1 | 19.5 | I | W |
| NUCLEAR | | | | | | |
| Hs.249247 | FBRNP | heterogeneous nuclear protein similar to rat helix | 10 | 31.4 | H | PC |
| | | destabitizing protein | | | | |

ONCOGENE

| | | | | | | |
|---|---|---|---|---|---|---|
| Hs.858 | RELB | v-rel avian reticuloendotheliosis viral oncogene homolog B | 19q13.2 | 260.5 | VH | W |
| | | (nuclear factor of kappa light polypeptide gene enhancer in | | | | |
| | | B-cells 3) | | | | |
| Hs.75569 | RELA | v-rel avian reticuloendotheliosis viral oncogene homolog A | 11q13 | 143.6 | VH | W |
| | | (nuclear factor of kappa light polypeptide gene enhancer in | | | | |
| | | B-cells 3 (p65)) | | | | |
| Hs.198951 | JUNB | jun B proto-oncogene | 19p13.2 | 56.9 | H | W |
| Hs.78465 | JUN | v-jun avian sarcoma virus 17 oncogene homolog | 1p32-p31 | 47.2 | H | W |
| Hs.300592 | MYBL1 | v-myb avian myeloblastosis viral oncogene homolog-like 1 | 8q22 | 43.0 | H | W |
| Hs.51305 | MAFF | v-maf musculoaponeurotic fibrosarcoma (avian) oncogene | 22q13.1 | 36.4 | H | PC |
| | | family, protein F | | | | |
| Hs.2780 | JUND | jun D proto-oncogene | 19p13.2 | 20.8 | I | W |
| *Hs.79070 | MYC | v-myc avian myelocytomatosis viral oncogene homolog | 8q24.12-q24.13 | 18.3 | I | W |
| Hs.85146 | ETS2 | v-ets avian erythroblastosis virus E26 oncogene homolog 2 | 21q22.2 | 11.0 | I | W |
| Hs.179718 | MYBL2 | v-myb avian myeloblastosis viral oncogene homolog-like 2 | 20q13.1 | 9.5 | L | W |
| Hs.92137 | MYCL1 | v-myc avian myelocytomatosis viral oncogene homolog 1, | 1p34.3 | 8.1 | L | W |
| | | lung carcinoma derived | | | | |
| Hs.724 | THRA | thyroid hormone receptor, alpha (avian erythroblastic | 17q11.2 | 6.2 | L | W |
| | | leukemia viral (v-erb-a) oncogene homolog) | | | | |
| Hs.2969 | SKI | v-ski avian sarcoma viral oncogene homolog | 1q22-q24 | 5.3 | L | W |
| Hs.110713 | DEK | DEK oncogene (DNA binding) | 6p23 | 4.9 | L | W |
| *Hs.157441 | SPI1 | spleen focus forming virus (SFFV) proviral integration | 11p11.2 | 4.5 | L | W |
| | | oncogene spil | | | | |
| Hs.181128 | ELK1 | ELK1, member of ETS oncogene family | Xp11.2 | 4.4 | L | W |
| Hs.431 | BMI1 | murine leukemia viral (bmi-1) oncogene homolog | 10p13 | 4.0 | L | W |
| Hs.1334 | MYB | v-myb avian myeloblastosis vital oncogene homolog | 6q22-q23 | 3.7 | L | W |
| Hs.252229 | MAFG | v-maf musculoaponeurotic fibrosarcoma (avian) oncogene | 17q25 | 3.5 | L | W |
| | | family, protein G | | | | |
| Hs.30250 | MAF | v-maf musculoaponeurotic fibrosarcoma (avian) oncogene | 16q22-q23 | 3.1 | L | W |
| | | homolog | | | | |
| PHD | | | | | | |
| Hs.166204 | PHF1 | PHD finger protein 1 | 6p21.3 | 7.2 | L | PC |

REPRESSOR

| Hs.144904 | NCOR1 | nuclear receptor co-repressor 1 | 17p11.2 | 21.2 | I | W |
|---|---|---|---|---|---|---|
| Hs.89421 | CIR | CBF1 interacting corepressor | 2p23.3-q24.3 | 6.7 | L | W |
| Hs.7222 | RBAK | RB-associated KRAB repressor | 7 | 6.0 | L | PC |
| Hs.5710 | CREG | cellular repressor of E1A-stimulated genes | 1q24 | 5.1 | L | PC |
| Hs.287994 | NCOR2 | nuclear receptor co-repressor 2 | 12q24 | 3.7 | L | W |
| RING | | | | | | |
| *Hs.14084 | RNF7 | ring finger protein 7 | 3q22-q24 | 401.9 | VH | PC |
| Hs.216354 | RNP5 | ring finger protein 5 | 6p21.3 | 396.4 | VH | N |
| Hs.97176 | RNF25 | ring finger protein 25 | 2p23.3-q34 | 358.7 | VH | N |
| Hs.59545 | RNF15 | ring finger protein 15 | 6p21.3 | 220.9 | VH | N |
| Hs.8834 | RNF3 | ring finger protein 3 | 4p16.3 | 58.4 | H | N |
| Hs.23794 | CHFR | checkpoint with forkhead and ring finger domains | 12 | 42.1 | H | PC |
| Hs.32597 | RNF6 | ring finger protein (C3H2C3 type) 6 | 13q12.2 | 31.5 | H | N |
| *Hs.6900 | RNF13 | ring finger protein 13 | 3p13-q26.1 | 10.4 | I | N |
| Hs.61515 | | Homo sapiens, Similar to ring finger protein 23, clone | N/A | 6.4 | L | N |
| | | MGC:2475, mRNA, complete cds | | | | |
| Hs.7838 | MKRN1 | makorin, ring finger protein, 1 | 7q34 | 5.9 | L | PC |
| Hs.35384 | RING1 | ring finger protein 1 | 6p21.3 | 4.4 | L | W |
| Hs.274295 | RNF9 | ring finger protein 9 | 6p21.3 | 4.3 | L | PC |
| Hs.59106 | CGR19 | cell growth regulatory with ring finger domain | 14q21.1-q23.3 | 4.2 | L | N |
| Hs.91096 | RNF | ring finger protein | 6p21.3 | 4.0 | L | N |
| OTHERS | | | | | | |
| Hs.326876 | SOX6 | Homo sapiens SOX6 mRNA, complete cds | 11p15.3 | 98.2 | H | W |
| Hs.185708 | EBF | early B-cell factor | 5q34 | 81.8 | H | W |
| Hs.288697 | MGC11349 | hypothetical protein MGC11349 | 3p13-q26.1 | 12.2 | I | N |
| Hs.23240 | | Home sampiens cDNA: FLJ21848 fis, clone HEP01925 | N/A | 11.6 | I | N |
| Hs.278270 | P23 | unactive progesterone receptor, 23 kD | 12 | 5.7 | L | PC |
| Hs.7367 | | Homo sapiens BTB domain protein (BDPL) mRNA, | N/A | 4.3 | L | PC |
| | | partial cds | | | | |
| The genes are presented by general category UniGene cluster identification number (ID), Gene Name and Gene Description are abstracted from UniGene (build version 135), Band = chromosomal band location; FB = level of expression as measured relative to background (fold over from background), as reported previously¹⁷. Abundance category is based on the relative expression level over | | | | | | |
| # background, using the following definitions: L, low level (>3-fold to <10-fold over background); I, intermediate level (≧10-fold to <25-fold); H, high level (>25-fold to <100-fold); and VH, very high level (≧100-fold). Characterization is based on a literature search, as described in the text: W, well-characterized; PC, partially-characterized; N, novel; N/A, not available. The | | | | | | |
| # asterisk (*) indicates genes which were also selected from the murine stem cell database analysis | | | | | | |

TABLE 4B
Human transcription factors identified by homology with murine transcription factors.

| | | | | | | | | |
|---|---|---|---|---|---|---|---|---|
| | | Human | | | | | | |
| | Mouse Gene | UniGene | Human | Human Gene | | | Abund- | Character- |
| Mouse Gene | Description | Cluster ID | Gene | Description | Band | FB | ance | ization |

| Nrf-1 | Activator involved in | Hs.180069 | NRF1 | nuclear respiratory factor | 7q32 | 425.8 | VH | W |
|---|---|---|---|---|---|---|---|---|
| | nuclear-mitochondrial | | | 1 | | | | |
| | interactions | | | | | | | |
| Dnmt-3b | De novo cytosine | Hs.251673 | DNMT3B | DNA (cytosine-5-)- | 20q11.2 | 336.2 | VH | W |
| | methyltransferase found | | | methyltransferase 3 beta | | | | |
| | in ES cells | | | | | | | |
| IFP-35 | associates with B-ATF | Hs.50842 | IFI35 | interferon-induccd protein | 17q21 | 191.4 | VH | PC |
| | | | | 35 | | | | |
| LL2in13291 | homolog of KIAA0326; | Hs.301094 | KIAA0326 | KIAA0326 protein | N/A | 126.4 | VH | N |
| | contains 19 C2H2 zinc | | | | | | | |
| | fingers | | | | | | | |
| Sox-13 | SRY-related; contains | Hs.201671 | SOX13 | SRY (sex determining | 1q32 | 73.3 | H | PC |
| | HMG box | | | region Y)-box 13 | | | | |
| SKIP | interacts with Ski, which | Hs.79008 | SNW1 | SKI-INTERACTING | 14q21.1- | 40.6 | H | W |
| | may arrest hematopoietic | | | PROTEIN | q24.3 | | | |
| | differentiation | | | | | | | |
| HPI- | Binds to TIF1 | Hs.142442 | HP1-BP74 | HP1-BP74 | 1pter- | 39.1 | H | N |
| BP74/hetero- | | | | | p36.13 | | | |
| chromatinic | | | | | | | | |
| LL2in10006 | Helicase and SNF2 | Hs.16933 | HARP | HepA-related protein | 2q34- | 28.6 | H | W |
| | domains; novel helicase | | | | q35 | | | |
| Stat-5a | Possible role in regulation | Hs.181112 | HSPC126 | HSPC126 protein | 13q12.2- | 25.4 | H | PC |
| | of endothelial function | | | | q13.3 | | | |
| CtBP2 | potent repressom; interacts | Hs.171391 | CTBP2 | C-terminal binding | 21q21.3 | 22.2 | I | W |
| | with Evi-1, AREB6, ZEB | | | protein 2 | | | | |
| | and FOG | | | | | | | |
| HMG-1 | Unwinds double-stranded | Hs.274472 | HMG1 | high-mobility group | 13q12 | 18.1 | I | W |
| | DNA | | | (nonhistone | | | | |
| | | | | chromosomal) protein 1 | | | | |
| HMG-17 | Alters interaction between | Hs.181163 | HMG17 | high-mobility group | 1p36.1 | 17.9 | I | W |
| | DNA and the histone | | | (nonhistone | | | | |
| | octamet, maintaining | | | chromosomal) protein 17 | | | | |
| | chromatin conformation | | | | | | | |
| LD5-1 | heterochromatosis locus | Hs.279586 | LOC51578 | adrenal gland protein AD- | 5 | 17.0 | I | N |
| | | | | 004 | | | | |
| Heterochroma- | regulated during cell | Hs.77254 | CBX1 | chromohox homolog 1 | 17q | 16.5 | I | PC |
| tin protein p25 | cycle | | | (Drosophila HP1 beta) | | | | |
| Dnmt-3a | De novo cytosine | Hs.241565 | DNMT3A | DNA (cytosinc-5-)- | 2p23 | 15.0 | I | W |
| | methyltransferase found | | | methyltransferase 3 alpha | | | | |
| | in ES cells | | | | | | | |
| SAP1a | Ets family; implicated in | Hs.169241 | ELK4 | ELK4, ETS-domain | 1q32 | 12.4 | I | W |
| | serum response of fos | | | protein (SRF accessory | | | | |
| | promoter | | | protein I) | | | | |
| Rpt-Ir | Down-regulates IL-2 | Hs.125300 | RNF21 | ring finger protein 21, | 11p15 | 11.7 | I | PC |
| | receptor | | | interferon-responsive | | | | |
| Histone H3.3A | nucleosomal histone | Hs.181307 | H3F3A | H3 histone, family 3A | 1q41 | 11.3 | I | W |
| HCNGP | Probably involved in | Hs.27299 | HCNGP | transcriptional regulator | 17 | 9.8 | L | N |
| | regulation of beta-2- | | | protein | | | | |
| | microglobulin genes | | | | | | | |
| HLF | bZip; fusion to E2A | Hs.250692 | HLF | hepatic leukemia factor | 17q22 | 8.7 | L | W |
| | results in B-lineage | | | | | | | |
| | leukemia; related to DBP | | | | | | | |
| WBSCR11 | Chr 11, Williams-Beuren | Hs.21075 | GTP2IRD1 | GTF2I repeat domain- | 7q11.23 | 8.5 | L | W |
| | Syndrome region; TFII-I | | | containing 1 | | | | |
| | domain | | | | | | | |
| P300/CBP co- | competes against TGIF to | Hs.225977 | NCOA3 | nuclear receptor | 20q12 | 8.2 | L | W |
| integrator | promote TGF-b- | | | coactivator 3 | | | | |
| | dependent transcriptional | | | | | | | |
| | activation | | | | | | | |
| TIP60 | Acetylates histones to | Hs.6364 | HTATIP | HIV-1 Tat interactive | 11 | 8.2 | L | W |
| | regulate X-chromosome | | | protein, 60 kDa | | | | |
| | dosage compensation | | | | | | | |
| CGGBP | Can bind the CGG | Hs.86041 | CGGBP1 | CGG triplet repeat | 3p12- | 7.2 | L | PC |
| | trinucleotide; may affect | | | binding protein 1 | p11.1 | | | |
| | FMR-1 promoter activity | | | | | | | |
| XE169 | Similar to jumonji ARID | Hs.283429 | SMCX | SMC (mouse) homolog, | Xp11.22- | 6.6 | L | W |
| | motif, 2 PHD fingers | | | X chromosome | p11.21 | | | |
| CPBP? | Core promoter element | Hs.285313 | COPEB | core promoter element | 10p15 | 6.5 | L | W |
| | bp? 3 C2H2 zinc fingers | | | binding protein | | | | |
| LL2in10261 | 7 C2H2 zinc fingers | Hs.278569 | SNX17 | sorting nexin 17 | 2p23- | 6.1 | L | N |
| | | | | | p22 | | | |
| SB1.8/DXS423E | chromosome segregation | Hs.211602 | SMC1L1 | SMC1 (structural | Xp11.22- | 6.0 | L | PC |
| | protein | | | maintenance of | p11.21 | | | |
| | | | | chromosomes 1, yeast)- | | | | |
| | | | | like 1 | | | | |
| HD1 | Histone deacetylase; | Hs.88556 | HDAC1 | histone deacetylase 1 | 1p34 | 5.9 | L | W |
| | binds to TGIF in a | | | | | | | |
| | complex that represses | | | | | | | |
| | TGF-b | | | | | | | |
| Erm | Ets-related | Hs.43697 | ETV5 | ets variant gene 5 (ets- | 3q28 | 5.8 | L | W |
| | | | | related molecule) | | | | |
| Ring-box | Component of VHL | Hs.279919 | RBX1 | ring-box 1 | 22q13.2 | 5.6 | L | PC |
| protein-1 | tumor suppressor | | | | | | | |
| | complex and SCF | | | | | | | |
| | ubiquitin ligase | | | | | | | |
| SPOP | Speckle-type nuclear | Hs.129951 | SPOP | speckle-type POZ protein | 17 | 5.5 | L | PC |
| | protein. BTB, Poz | | | | | | | |
| | domains | | | | | | | |
| NAB-1 | repressor of Krox20; May | Hs.107474 | NAB1 | NGFI-A binding protein 1 | 2q32.3- | 5.4 | L | W |
| | repress proliferation, | | | (ERG1 binding protein 1) | q33 | | | |
| | differentiation | | | | | | | |
| Prox1 | Homeobox transcription | Hs.110803 | LOC51637 | CGI-99 protein | 14q13.1- | 5.4 | L | N |
| | factor required for | | | | q13.3 | | | |
| | lymphatic development | | | | | | | |
| Nmi | Interacts with myc, max, | Hs.54483 | NMI | N-myc (and STAT) | 2p24.3- | 4.5 | L | W |
| | and fos | | | interactor | q21.3 | | | |
| TAK1/TR4 | orphan nuclear receptor; | Hs.520 | NR2C2 | nuclear receptor | 3p25 | 4.2 | L | W |
| | contains C4 zinc finger | | | subfamily 2, group C, | | | | |
| | | | | member 2 | | | | |
| LL2in14617 | Homologous human | Hs.238954 | | ESTs, Weakly similar to | N/A | 4.2 | L | W |
| | uncharacterized protein | | | KIAA1204 protein | | | | |
| | USF1 contains HLH | | | [H. sapiens] | | | | |
| | signature | | | | | | | |
| LL2in10596 | KIAA0244; contains | Hs.78893 | KIAA0244 | KIAA0244 protein | 6q12 | 3.8 | L | N |
| | TFHS and PHD motifs | | | | | | | |
| Pilot/EGR-3 | Zinc finger protein | Hs.74088 | EGR3 | early growth response 3 | 8p23- | 3.8 | L | W |
| | | | | | p21 | | | |
| CHD-1 | contains chromodomain | Hs.22670 | CHD1 | chromodomain helicase | 5q15- | 3.7 | L | W |
| | | | | DNA binding protein 1 | q21 | | | |
| HUNKI | Contains two bromo | Hs.278675 | BRD4 | bromodomain-containing | 19p13.1 | 3.7 | L | PC |
| | domains | | | 4 | | | | |
| LL1-46 | KIAA0518; HLH domain | Hs.23763 | MGA | Max-interacting protein | 15q15 | 3.4 | L | N |
| Nrf-2 | A relative of kelch | Hs.155396 | NFE2L2 | nuclear factor (erythroid- | 2q31 | 3.4 | L | W |
| | suppresses nrf-2 function | | | derived 2)-like 2 | | | | |
| homeodomain- | phosphorylates | Hs.236131 | HIPK2 | homeodomain-interacting | 7q32- | 3.3 | L | PC |
| interact. pk 2 | homeodomain | | | protein kinase 2 | q34 | | | |
| | transcription factors | | | | | | | |
| SWI-SNF (60 | opposes chromatin- | Hs.79335 | SMARCD1 | SWI/SNF related, matrix | 12q13- | 3.2 | L | PC |
| kDa subunit) | dependent repression of | | | associated, actin | q14 | | | |
| | transcription | | | dependent regulator of | | | | |
| | | | | chromatin, subfamily d, | | | | |
| | | | | member 1 | | | | |
| The table presents the murine gene name, the murine UniGene description, the Human UniGene Cluster ID, the name and UniGene description of the human gene, and the chromosome band (Band). FB = level of expression as measured relative to background (fold over background), as reported previously. 17 Abundance category is based on the relative | | | | | | | | |
| # expression level over background, using the following definitions: L, low level (≧3-fold to <10-fold over background); I, intermediate level (≧10-fold to <25-fold); H, high level (≧25-fold to <100-fold); and VH, very high level (≧100-fold). Characterization is based on a literature search, as described | | | | | | | | |
| # in the text: W, well-characterized; PC, partially-characterized; N, novel; N/A, not available. | | | | | | | | |

TABLE 5
Novel transcription factors identified by database search.

| Unigene | Gene | | Character- | | |
|---|---|---|---|---|---|
| Cluster ID | Name | Gene Description | ization | Available Function/Information | References |
| Hs.2815 | POU6F1 | POU domain, class 6, | PC | Member of the class IV POU homeodomain | Wey E and Schafer |
| | | transcription factor 1 | | family of transcription factors. | BW Biochem Biophys |
| | | | | | Res Commun. 1996 |
| Hs.239720 | CNOT2 | CCR4-NOT transcription | PC | may function as a transcription factor. | Albert TK et al. |
| | | complex, subunit 2 | | | Nucleic Acids Res. |
| | | | | | 2000. |
| Hs.108106 | ICBP90 | transcription factor | PC | CCAAT binding protein, maybe involved in the | Hopfner R, et al. Gene. |
| | | | | regulation of topoisomerase IIalpha gene | 2001. |
| | | | | expression. | |
| Hs.294101 | PBX3 | pre-B-cell leukemia | PC | Member of the homeodomain family of DNA- | Knoepfler PS and |
| | | transcription factor 3 | | binding proteins; very strongly similar to | Kamps MP. Mech |
| | | | | murine Pbx3. | Dev. 1997 |
| Hs.249184 | TCF19 | transcription factor 19 | PC | Putative transcription factor; may be involved | Teraoka Y et al. Tissue |
| | | (SCI) | | in the later stages of cell cycle progression. | Antigens. 2000. |
| Hs.29417 | ZF | HCF-binding | PC | Contains a basic domain-leucine zipper (bZIP) | Lu R and Misra V |
| | | transcription factor | | region, an acidic activation domain and a | Nucleic Acids Res. |
| | | Zhangfei | | consensus HCF (host cell factor)-binding motif | 2000. |
| Hs.155313 | DATF1 | death associated | PC | protein has two Zn finger motifs, nuclear | Garcia-Domingo D, et |
| | | transcription factor 1 | | localization signals, and transcriptional | al. Proc. Nat. Acad. |
| | | | | activation domains; maybe involved in cell | Sci. 1999. |
| | | | | death during development. | |
| Hs.26703 | CNOT8 | CCR4-NOT transcription | PC | Similar to S. cerevisise transcriptional regulator | Albert TK, et al. |
| | | complex, subunit 8 | | Pop2p. The yeast CCR4-NOT protein complex | Nucleic Acids Res. |
| | | | | is a global regulator of RNA polymerase II | 2000. |
| | | | | transcription. | |
| Hs.78061 | TCF21 | transcription factor 21 | PC | involved in epithelial-mesenchymal interactions | Robb L, et al Dev |
| | | | | in kidney and lung morphogenesis, and may | Dyn. 1998. |
| | | | | play a role in the specification or (differentiation | |
| | | | | of one or more subsets of epicardial cell types. | |
| Hs.59506 | DMRT2 | doublesex and mab-3 | PC | May be involved in male sexual development; | Ottolenghi C, et al. |
| | | related transcription | | contains a DNA-binding domain. | Genomics 2000. |
| | | factor 2 | | | |
| Hs.21704 | TCF12 | transcription factor 12 | PC | expressed in many tissues, and may participate | Di Rocco G, et al. Mol |
| | | (HTF4, helix-loop-helix | | in regulating lineage-specific gene expression | Cell Biol. 1997. |
| | | transcription factors 4) | | through the formation of heterodimers with | |
| | | | | other bHLH E-proteins. | |
| Hs.226318 | CNOT7 | CCR4-NOT transcription | PC | The protein encoded by this gene binds to an | Prevot D, et al. J Biol |
| | | complex, subunit 7 | | anti-proliferative protein, B-cell translocation | Chem 2001 |
| | | | | protein 1, which negatively regulates cell | |
| | | | | proliferation | |
| Hs.35841 | NFIX | nuclear factor I/X | PC | The nuclear factor I (NFI) family of | Fletcher CF, et al |
| | | (CCAAT-binding | | transcription/replication proteins is requiied for | Mamm Genome. 1999. |
| | | transcription factor) | | the cell type-specific expression of a number of | |
| | | | | cellular and viral genes. | |
| Hs.100932 | TCF17 | transcription factor 17 | PC | a human homologue of rat zinc finger gene Kid | Przyborski SA, et al |
| | | | | 1; contains a Kruppel-associated box (KRAB) | Cancer Res. 1998. |
| | | | | and C2H2 zinc fingers. | |
| Hs. 92282 | PITX2 | paired-like homeodomain | PC | may regulate gene expression and control cell | Degar BA, et al. Exp |
| | | transcription factor 2 | | differentiation; member of the homeodomain | Hematol. 2001. |
| | | | | family of DNA binding proteins F. | |
| Hs.97624 | HSF2BP | heat shock transcription | PC | HSF2 binding protein (HSF2BP) associates | Yoshima T et al. Gene |
| | | factor 2 binding protein | | with HSF2. HSF2BP may therefore be involved | 1998. |
| | | | | in modulating HSF2 activation. | |
| Hs.20423 | CNOT4 | CCR4-NOT transcription | PC | The yeast CCR4-NOT protein complex is a | Albert, T. K. et al |
| | | complex, subunit 4 | | global regulator of RNA polymerase II | Nucleic Acids Res. |
| | | | | transcription. | 2000. |
| Hs.2430 | TCFL1 | transcription factor-like 1 | PC | may function as a transcription factor | Horikawa J, et al |
| | | | | | Biochem. Biophys. |
| | | | | | Res. Commun. 1995. |
| Hs.30824 | LZTFL1 | leucine zipper | N | The LZTFL1 gene has two transcript isoforms | Kiss H, et al. |
| | | transcription factor-like 1 | | displaying alternative polyadenylation. | Genomics. 2001. |
| Hs.27299 | HCNGP | transcriptional regulator | N | Strongly similar to uncharacterized murine | N/A |
| | | protein | | Hcngp | |
| Hs.93748 | | Homo sapiens cDNA | N | N/A | N/A |
| | | clone moderately similar | | | |
| | | to Transcription Factor | | | |
| | | BTF3 | | | |
| Hs.173854 | PAXIPIL | PAX transcription | N | N/A | N/A |
| | | activation domain | | | |
| | | interacting protein 1 like | | | |
| Hs.268115 | | ESTs, Weakly similar to | N | N/A | N/A |
| | | T08599 probable | | | |
| | | transcription factor | | | |
| | | CA150 [H sapiens] | | | |
| Hs.24572 | | ESTs, Weakly similar to | N | N/A | N/A |
| | | TC17_HUMAN | | | |
| | | TRANSCRIPTION | | | |
| | | FACTOR 17 [H. sapiens] | | | |
| Hs.171185 | P38IP | transcription factor (p38 | N | N/A | N/A |
| | | interacting protein) | | | |
| The table presents 25 novel and poorly-characterized genes or ESTs resulting from these studies which are likely to be transcription factors. The table presents the UniGene Cluster Identification (ID) number, the gene name, the UniGene Description of the sequence, the literature characterization, additional available functional information | | | | | |
| # about the gene, and the literature citation. Characterization based on literature search is described in the text as: W, well characterized; PC, partially characterized; N, novel; N/A, not available. | | | | | |

We claim:

1. A database comprising the nucleotide sequences of a plurality of cDNA molecules selected from human CD34 antigen positive hematopoictic cells, said database useful for the analysis of hematopoietic tissue, said tissue selected from the group consisting of bone marrow, peripheral blood, stem cells, transplanted marrow, and leukemia cells from human and related primates including baboon.

2. The database of claim 1 comprising molecules having the nucleotide sequences designated by the unique identifiers as shown in Table 2.

3. A microchip comprising the database of claim 1 or a subset thereof.

4. A method for selecting a database containing expressed genes from primate CD34 antigen positive cells, said method comprising:
(a) selecting genes expressed in human cells; and
(b) further selecting genes selected in (a) whose expression levels are similar between humans and baboons.

5. The method of claim 4, wherein expression above background in human cells is at least >3-fold.

6. The method of claim 4, wherein the expression level differs between baboons and humans by 3 fold or less.

7. The method of claim 4, wherein the expression levels are greater than or equal to 7-fold above background in human cells.

8. The method of claim 4, wherein gene expression is measured by the gene filter method.

9. A computer system comprising:
(a) a database containing nucleotide sequences pertaining to a plurality of nucleotide sequences selected in accord with the method of claim 4;
(b) a first hierarchy of function categories into which at least some of said sequences are grouped; and
(c) a user interface allowing a user to selectively view information regarding said plurality of said sequences as it relates to said first hierarchy.

10. The computer system of claim 9, wherein the sequences are selected from the group consisting of ESTs, full-length sequences, and combinations thereof.

11. The computer system of claim 9, wherein the user interface allows the user to selectively view information regarding a subset of said plurality of said sequences, which subset is grouped in both a selected category and for a selected application.

12. A computer-implemented method for managing information relating to hematopoietic analyses said method comprising:
(a) a first identifier identifying a target sample applied to a probe array;
(b) a second identifier identifying said probe array to which said target sample was applied; and
(c) creating an electronically-stored array table, said table storing a record for said polymer probe array, said record comprising
(i) a plurality of fields storing at least one of a plurality of data identifiers, including,
(ii) said second identifier identifying said probe array, and
(iii) a third identifier specifying a layout of probes on said probe array.

13. The computer-implemented method for managing information of claim 12, wherein the probe array is on a chip.

14. A database method for analyzing hematopoetic tissue said method comprising:
(a) providing a first database comprising a first plurality of records, one for each of a plurality of cDNA sequences, said records having at least one of a plurality of fields storing:
(i) a first attribute identifying a target sample applied to a probe array;
(ii) a second attribute identifying said probe array to which said target sample was applied; and
(b) providing a second database comprising a second plurality of records for said probe array, said records having at least one of a plurality of fields storing:
(i) said second attribute identifying said probe array; and
(ii) a third attribute specifying a layout of probes on said probe array.

15. The database method for analyzing gene expression information of claim 14, wherein said first database and said second database are relational database tables.

16. The database method of claim 14, wherein the array is on a chip.

17. A method of compiling a database of human cDNA sequences that have functions suitable for a specific purpose from a CD34+ transcriptosome database, said method comprising:
(a) searching functional descriptors associated with gene-oriented clusters of the CD34+ transcriptosome database for descriptors related to the suitable functions;
(b) selecting gene-oriented clusters that include at least one of the suitable descriptors related to the suitable functions;
(c) cross-referencing a murine database with the human database to confirm sequences in the database;
(d) removing redundant cDNA sequences within the selected clusters; and
(e) searching a database of clone sequences with the cDNA sequence having the highest level of expression within each selected cluster, to verify homology.

18. The method of claim 17, wherein the CD34+ database is that at http://westsunhema.uic.edu/cd34.html, the suitable functions are those characteristic of transcription factors and their regulatory proteins, wherein the regulatory proteins comprise co-repressors and co-activators, nuclear factors, and other DNA-interacting proteins, and wherein the functional descriptors are from UniGene (http://www.ncbi.nlm.nih.gov/UniGene/) and the data base of clone sequences is Genbank.

19. A human transcription factor/regulatory protein database as listed in Table 5.
